(19) United States
(12) Patent Application Publication
(10) Pub. No.: US 2013/0129686 A1
(43) Pub. Date: May 23, 2013

(54) Reducing Inflammation Using Cell Therapy

(71) Applicants: Steven L. Highfill, Rockville, MD (US); Jakub Tolar, Minneapolis, MN (US); Bruce M. Blazar, Golden Valley, MN (US)

(72) Inventors: Steven L. Highfill, Rockville, MD (US); Jakub Tolar, Minneapolis, MN (US); Bruce M. Blazar, Golden Valley, MN (US)

(73) Assignee: Regents of the University, St Paul, MN (US)

(21) Appl. No.: 13/510,491

(22) PCT Filed: Nov. 19, 2009
(86) PCT No.: PCT/US09/65128

§ 371 Date: Nov. 19, 2012

(51) Int. Cl. A61K 35/12

(52) U.S. Cl. CPC A61K 35/12
USPC 424/93.7

(57) ABSTRACT

The invention provides methods for treating pathological conditions associated with an undesirable inflammatory component, including graft-versus-host disease. The invention is generally directed to reducing inflammation by administering cells that express and/or secrete prostaglandin E2 (PGE2). The invention is also directed to drug discovery methods to screen for agents that modulate the ability of the cells to express and/or secrete PGE2, such as PGE2 receptor agonists. The invention is also directed to cell banks that can be used to provide cells for administration to a subject, the banks comprising cells having desired levels of PGE2 expression and/or secretion. The invention is also generally directed to delivering cells directly to lymphohematopoietic tissue, such as spleen, lymph nodes, and bone marrow. The invention is, thus, also directed to a method for treating inflammation by administering cells directly into sites of lymphohematopoiesis, such as spleen, lymph nodes, and bone marrow. The administered cells include those that reduce the activation and/or proliferation of T-cells. Such cells may or may not express and/or secrete PGE2.

### FIELD OF THE INVENTION

The invention provides methods for treating pathological conditions associated with an undesirable inflammatory component, including graft-versus-host disease. The invention is generally directed to reducing inflammation by administering cells that express and/or secrete prostaglandin E2 (PGE2). The invention is also directed to drug discovery methods to screen for agents that modulate the ability of the cells to express and/or secrete PGE2, such as PGE2 receptor agonists. The invention is also directed to cell banks that can be used to provide cells for administration to a subject, the banks comprising cells having desired levels of PGE2 expression and/or secretion. The invention is also directed to compositions comprising cells of specific desired levels of PGE2 expression and/or secretion, such as pharmaceutical compositions. The invention is also directed to diagnostic methods conducted prior to administering the cells to a subject to be treated, including assays to assess the desired potency of the cells to be administered. The invention is further directed to post-treatment diagnostic assays to assess the effect of the cells on a subject being treated. In one embodiment, such cells are stem cells or progenitor cells. The cells may have pluripotent characteristics. These may include expression of pluripotentiality markers and broad differentiation potential. In one specific embodiment, cells are non-embryonic non germ-cells that express markers of pluripotentiality and/or broad differentiation potential.

The invention is also generally directed to delivering cells directly to the site of initial T-cell allopriming, such as spleen, lymph nodes, and bone marrow. The invention is, thus, also directed to a method for treating inflammation by administering cells directly into sites of lymphohematopoiesis, such as spleen, lymph nodes, and bone marrow. The administered cells include those that reduce the activation and/or proliferation of T-cells. Such cells may or may not express and/or secrete PGE2. In one embodiment, such cells are stem cells or progenitor cells. Such cells may or may or may not express and/or secrete PGE2. In one specific embodiment, cells are non-embryonic non germ-cells that express markers of pluripotentiality and/or broad differentiation potential.

### BACKGROUND OF THE INVENTION

### Inflammation

Inflammation can be classified as acute or chronic. Acute inflammation is the initial response of the body to harmful stimuli and is achieved by the increased movement of plasma and leukocytes from the blood into the injured tissues. It occurs as long as the injurious stimulus is present and ceases once the stimulus has been removed, broken down, or walled off by scarring (fibrosis). A cascade of biochemical events propagates and matures the inflammatory response, involving the local vascular system, the immune system, and various cells within the injured tissue. Once in the tissue, leukocytes migrate along a chemotactic gradient to reach the site of injury, where they can attempt to remove the stimulus and repair the tissue. Meanwhile, several biochemical cascade systems, consisting of chemicals known as plasma-derived inflammatory mediators, act in parallel to propagate and mature the inflammatory response. These include the complement system, coagulation system and fibrinolysis system. Finally, down-regulation of the inflammatory response concludes acute inflammation.

Prolonged inflammation, known as chronic inflammation, leads to a progressive shift in the type of cells at the site of inflammation and is characterised by simultaneous destruction and healing of the tissue from the inflammatory process. Chronic inflammation is a pathological condition characterised by concurrent active inflammation, tissue destruction, and attempts at repair. Chronic inflammation is not characterised by the classic signs of acute inflammation listed above. Instead, chronically inflamed tissue is characterised by the infiltration of mononuclear immune cells (monocytes, macrophages, lymphocytes, and plasma cells), tissue destruction, and attempts at healing, which include angiogenesis and fibrosis.

### Prostaglandin E2 (PGE2)

Prostanoids are a group of lipid mediators that regulate numerous processes in the body. These processes include regulation of blood pressure, blood clotting, sleep, labor and inflammation. When tissues are exposed to diverse physiological and pathological stimuli, arachidonic acid is liberated from membrane phospholipids by phospholipase A2 and is converted to PGH2 by prostaglandin H synthase (PGHS; also termed cyclooxygenase COX). PGH2, is the common substrate for a number of different synthases that produce the major prostanoids including PGD2, PGE2, prostacyclin (PGI2) and tromboxane (TXA2).

Among these, PGE2 plays crucial roles in various biological events such as neuronal function, female reproduction, vascular hypertension, tumorigenesis, kidney function and inflammation (Kobayashi, T. et al., Prostaglandin and Other Lipid Mediat, 68-69:557-574 (2002); Harris, S. G. et al., Trends in Immunol, 23:144-150 (2002)).

PGE2 is synthesized in substantial amounts at sites of inflammation where it acts as a potent vasodilator and synergistically with other mediators, such as histamine and bradykinin, causes an increase in vascular permeability and edema (Davies, P. et al., Annu Rev Immunol, 2:335-357 (1984)). Moreover PGE2 is a central mediator of febrile response triggered by the inflammatory process and intradermal PGE2 is hyperalgesic in the peripheral nervous system (Dinarello et al., Curr Biol, 9:147-150 (1999)).

PGE2 has been implicated in the development of inflammatory symptoms and cytokine production in vivo. For example, selective neutralization of PGE2 was found to block inflammation, hyperalgesia, and interleukin-6 (IL-6) production in vivo, using a neutralizing anti-PGE2 monoclonal antibody, 2B5. See Portanova et al., J Exp Med, 184:883 (1996).

PGE2 can act through at least four different receptors (EP1-4) and the regulation of expression of the various subtypes of EP receptors on cells by inflammatory agents, or even PGE2 itself, enables PGE2 to affect tissues in a very specific manner (Narumiya S. et al., J Clin Invest, 108:25-30 (2001)).

The receptors are rhodopsin-type receptors containing seven transmembrane domains coupled through the intracellular sequences to specific G-proteins with different second messenger signaling pathways. See Harris et al. above. PGE2 has diverse effects on regulation and activity of T-cells. The inhibition of T-cell proliferation by PGE2 has been well established. The effects of PGE2 on the apoptosis of T-cells depends on the maturity and activation state of the T-cell. PGE2 also has an effect on the production of cytokines by T-cells, for example, by inhibiting cytokines such as interferon-γ (IFN-γ) and IL-2 by Th-1 cells. PGE2, however, also has a Th2-inducing activity on T-cells. It enhances the production of Type 2 cytokines and antibodies. It acts on T-cells to enhance production of IL-4, IL-5, and IL-10, but inhibits production of IL-2 and IFN-γ. Acting on B-cells, PGE2 stimulates isotype class switching to induce the production of IgG-1 and IgE. On antigen-presenting cells, such as macrophages and dendritic cells, PGE2 induces expression of IL-10 and inhibits the expression of IL-12, IL-12 receptor, TNF-α, and IL-1β. The overall result is an enhancement of Th-2 responses and inhibition of Th-1 responses. See Harris et al. (above).

### Resolution of Acute Inflammation and PGE2

Eventually, immune cells disappear from a previously active site of inflammation. The initial injury is thought to be related to the resolution of inflammation. The quelling of inflammation is the result of a specific sequence of events set in motion at the beginning of an inflammatory attack. To investigate the sequence, experiments have been performed where inflammation was created in a small air pouch on the back of a mouse. See Serhan, C. et al., Nature Immunology, 2:612-619 (2001). As neutrophils entered the site of inflammation, they produced leukotriene B4 (LTB4), which recruited more cells into the pouch. Neutrophils also produced cyclooxygenase-2 (COX-2), which led to the production of PGE2. PGE2 caused a switch from a pro-inflammatory to an anti-inflammatory strategy: 15-lipoxygenase (15-LO) was induced, leading to lipoxin A4 (LXA4) production. Soon after, the flow of new immune cells dropped and inflammation eventually resolved.

To ascertain whether the PGE2 might be causing the switch to LXA4, the researchers mixed it with the LTB4-producing immune cells. The cells then began producing an enzyme required for lipoxin. Deprived of prostaglandin, the cells could not produce the enzyme. To confirm that LXA4, which is produced by neutrophils themselves, plays a role in resolution, they examined the chest cavity fluids of patients with and without inflammatory disease. Only those with inflammatory disease displayed LXA4 activity. To discover how and when the neutrophils were producing lipoxin and the other inflammatory agents, the researchers monitored the rise and fall of each in the pouched mice. They found that the consecutive production of LTB4, PGE2, and LXA4 corresponded to the waxing and waning of immune cells. Further experiments confirmed that LTB4 and LXA4 were responsible, on the one hand, for inciting inflammation and, on the other, for dampening it.

The way the cells make the switch between the two tactics is by turning on prostaglandin production. PGE2 works by inducing the enzyme required for lipoxin production. This enzyme, 15-lipoxygenase (15-LO), is found naturally in the neutrophils of patients with chronic inflammatory disease. That function is not present in circulating neutrophils from healthy donors.

### Graft-Versus-Host-Disease (GVHD)

Recipients of allogeneic transplants often experience acute GVHD due to alloreactive T-cells present in the allograft. GVHD involves a pathophysiology that includes host tissue damage, increased secretion of proinflammatory cytokines (TNF-α, IFN-γ, IL-1, IL-2, IL-12), and the activation of dendritic cells and macrophages, NK cells, and cytotoxic T-cells.

### SUMMARY OF THE INVENTION

The invention is broadly directed to a method for reducing inflammation.

The invention is more specifically directed to a method to reduce T-cell activation and/or proliferation, including, but not limited to, allogeneic T-cells.

The invention is more specifically directed to a method to reduce pro-inflammatory cytokine production, including, but not limited to, in T-cells and antigen-presenting cells.

The invention is also directed to a method to alter the balance away from positive stimulatory and toward negative inhibitory co-stimulatory pathway expression in T-cells and antigen-presenting cells.

The invention is also directed to a method to reduce GVHD-induced injury, including GVHD-induced lethality.

The invention is also directed to a method for providing prostaglandin E2 (PGE2) to achieve any of the above results.

Pro-inflammatory cytokines include, but are not limited to, TNF-α, IL-1, IL-2, IL-12, amphiregulin, LPS and other Toll-like receptor ligands (pathogenic peptides such as fMLP, peptides from damaged tissues, such as fibronectin fragments) thrombin, histamines, oxygen radicals, and IFN-γ.

T-cells include CD4⁺, CD8⁺, γδT-cells, and natural killer cells.

According to this invention, all of the above effects (i.e., reducing inflammation, providing PGE2, etc.) can be achieved by administering cells expressing and/or secreting PGE2 or medium conditioned by the cells. Cells include, but are not limited to, non-embryonic non-germ cells having characteristics of embryonic stem cells, but being derived from non-embryonic tissue, and expressing and/or secreting PGE2. Such cells may be pluripotent and express pluripotency markers, such as one or more of oct4, telomerase, rex-1, rox-1, sox-2, nanog, SSEA-1 and SSEA-4. Other characteristics of pluripotency can include the ability to differentiate into cell types of more than one germ layer, such as two or three of ectodermal, endodermal, and mesodermal embryonic germ layers. Such cells may or may not be immortalized or transformed in culture. Further, they may or may not be tumorigenic, such as not producing teratomas. If cells are transformed or tumorigenic, and it is desirable to use them for infusion, such cells may be disabled so they cannot form tumors in vivo, as by treatment that prevents cell proliferation into tumors. Such treatments are well known in the art. Such cells may naturally express/secrete PGE2 or may be genetically or pharmaceutically modified to enhance expression and/or secretion.

In view of the property of the PGE2-expressing cells to achieve the above effects, the cells can be used in drug discovery methods to screen for an agent that modulates the ability of the cells to express and/or secrete PGE2 so as to be able achieve any of the above effects. Such agents include, but are not limited to, small organic molecules, antisense nucleic acids, siRNA DNA aptamers, peptides, antibodies, non-antibody proteins, cytokines, chemokines, and chemo-attractants.

Because the effects described in this application can be caused by secreted PGE2, not only the cells, but also conditioned medium produced from culturing the cells, is useful to achieve the effects. Such medium would contain the secreted factor and, therefore, could be used instead of the cells or added to the cells. So, where cells can be used, it should be understood that conditioned medium would also be effective and could be substituted or added.

In view of the property of the PGE2-expressing cells to achieve the above effects, cell banks can be established containing cells that are selected for having a desired potency to express and secrete PGE2 so as to be able to achieve any of the above effects. Accordingly, the invention encompasses assaying cells for the ability to express and/or secrete PGE2 and banking the cells having a desired potency. The bank can provide a source for making a pharmaceutical composition to administer to a subject. Cells can be used directly from the bank or expanded prior to use.

Accordingly, the invention also is directed to diagnostic procedures conducted prior to administering the cells to a subject, the pre-diagnostic procedures including assessing the potency of the cells to express and/or secrete PGE2 so as to be able to achieve one or more of the above effects. The cells may be taken from a cell bank and used directly or expanded prior to administration. In either case, the cells would be assessed for the desired potency. Or the cells can be derived from the subject and expanded prior to administration. In this case, as well, the cells would be assessed for the desired potency prior to administration.

Although the cells selected for PGE2 expression are necessarily assayed during the selection procedure, it may be preferable and prudent to again assay the cells prior to administration to a subject for treatment to ensure that the cells still express desired levels of PGE2. This is particularly preferable where the expressor cells have been stored for any length of time, such as in a cell bank, where cells are most likely frozen during storage.

With respect to methods of treatment with cells expressing/secreting PGE2, between the original isolation of the cells and the administration to a subject, there may be multiple (i.e., sequential) assays for PGE2 expression. This is to ensure that the cells still express/secrete PGE2 after manipulations that occur within this time frame. For example, an assay may be performed after each expansion of the cells. If cells are stored in a cell bank, they may be assayed after being released from storage. If they are frozen, they may be assayed after thawing. If the cells from a cell bank are expanded, they may be assayed after expansion. Preferably, a portion of the final cell product (that is physically administered to the subject) may be assayed.

The invention further includes post-treatment diagnostic assays, following administration of the cells, to assess efficacy. The diagnostic assays include, but are not limited to, analysis of inflammatory cytokines and chemokines in the patient's serum, blood, tissue, etc.

The invention is also directed to a method for establishing the dosage of such cells by assessing the potency of the cells to express and/or secrete PGE2 so as to be able to achieve one or more of the above effects.

The invention is also directed to compositions comprising a population of the cells having a desired potency, and, particularly the expression and/or secretion of desired amounts of PGE2. Such populations may be found as pharmaceutical compositions suitable for administration to a subject and/or in cell banks from which cells can be used directly for administration to a subject or expanded prior to administration.

The methods and compositions of the invention are useful for treating any disease involving inflammation. This includes, but is not limited to, acute and chronic conditions in cardiovascular, e.g., acute myocardial infarction; central nervous system injury, e.g., stroke, traumatic brain injury, spinal cord injury; peripheral vascular disease; pulmonary, e.g., asthma, ARDS; autoimmune, e.g., rheumatoid arthritis, multiple sclerosis, lupus, sclerodoma; psoriasis; gastrointestinal, e.g., graft-versus-host-disease, Crohn's disease, diabetes, ulcerative colitis, acute and chronic transplantation rejection, and dermatitis.

In one particular embodiment, the methods and compositions of the invention are used to treat GVHD. For this treatment, one would administer the cells expressing PGE2. Such cells would have been assessed for the amount of PGE2 that they express and/or secrete and selected for desired amounts of PGE2 expression and/or secretion.

It is understood, however, that for treatment of any of the above diseases, it may be expedient to use such cells; that is, one that has been assessed for PGE2 expression and/or secretion and selected for a desired level of expression and/or secretion prior to administration for treatment of the condition.

The invention is also directed to achieving any of the above treatments by means of methods of directing cells to lymphoid tissue, such as secondary lymphoid tissue, using lymphatic delivery routes or selecting donor cells with optimal homing properties or inducing homing receptors on therapeutic cells by small molecule or biological preconditioning or by coating cells with receptors to direct increased retention.

In one embodiment, cells are delivered directly to the lymphohematopoietic system in so that T-cells are directly exposed to the administered cells at these sites. Sites include spleen, lymph node, bone marrow, Peyer's patches, gastrointestinal lymphoid tissue (GALT), bronchus associated lymphoid tissue (BALT), and thymus. The lymphoid tissue may be primary, secondary or tertiary depending upon the stage of lymphocyte development and maturation it is involved in. Primary (central) lymphoid tissues serve to generate mature virgin lymphocytes from immature progenitor cells. The thymus and the bone marrow constitute the primary lymphoid tissues involved in the production and early selection of lymphocytes. Secondary lymphoid tissue provides the environment for the foreign or altered native molecules (antigens) to interact with the lymphocytes. It is exemplified by the lymph nodes, and the lymphoid follicles in tonsils, Peyer's patches, spleen, adenoids, skin, etc., that are associated with the mucosa-associated lymphoid tissue (MALT). The tertiary lymphoid tissue typically contains far fewer lymphocytes, and assumes an immune role only when challenged with antigens that result in inflammation. It achieves this by importing the lymphocytes from blood and lymph.

Cells delivered by the above directed methods of administration include cells that express PGE2 and cells that do not express PGE2. The methods can generally apply to any cell that reduces the activation and/or proliferation of T-cells. The cells may include stem or progenitor cells, including those described in this application. In one embodiment, the cells are non-embryonic, non-germ cells that express pluripotentiality markers, e.g., one or more of telomerase, rex-1, sox-2, oct4, rox-1, and/or have broad differentiation potential, e.g., at least two of ectodermal, endodermal, and mesodermal cell types. Delivering such cells by means of the above directed route can be used generally to treat inflammation, including but not limited to, any of the disorders disclosed herein. In one embodiment, the pathology is GVHD and the route is intra-splenic. In a highly specific embodiment, the pathology is GVHD, the route is intra-splenic, and the cells are non-embryonic, non-germ cells that express pluripotentiality markers, e.g., one or more of telomerase, rex-1, sox-2, oct4, rox-1, and/or have broad differentiation potential, e.g., at least two of ectodermal, endodermal, and mesodermal cell types.

For this directed route, method involving PGE2 expression in the cells may result from expression of an endogenous cellular gene in a recombinant or non-recombinant cell or may result from expression of an exogenously-introduced partial or full PGE2 coding sequence. Accordingly, the method may be performed with virtually any cell known in the art that could serve as a recombinant host, in addition to those cells that naturally express PGE2 (i.e., non-recombinant with respect to PGE2 expression).

With respect to delivery directly to lymphohematopoietic tissues, the invention may exclude cells that activate or cause proliferation of T-cells, such as dendritic cells. CD34⁺ hematopoietic stem cells may also be excluded.

### BRIEF DESCRIPTION OF THE FIGURES

FIG. 1—MAPC potently inhibit allogeneic T cell proliferation and activation. A MLR reaction was performed by mixing B6 purified T-cells with irradiated BALB/c stimulators (1:1) and B6 MAPCs (1:10, 1:100)(A). These cultures were pulsed with ³H-thymidine on the indicated days harvested 16 hours later. Proliferation was determined as a measure of radioactive uptake. A MLR reaction was performed as above using BALB/c T-cells plus B6 stimulators and BALB/c MAPC (B), or BALB/c T-cells plus B10.Br stimulators and B6 MAPCs (C). FACS analysis of B6>BALB/c MLR+B6 MAPC was performed on the indicated days and gated on CD4+ T-cells (D) or CD8+ T-cells (E) in conjunction with activation markers.

FIG. 2—MAPC-mediated suppression in vitro is independent of Tregs. A B6>BALB/c MLR culture was performed using purified T-cells or T-cells that were CD25-depleted and MAPCs at 1:10 ratios. ³H-thymidine was added on the indicated days and proliferation was measured (A). FACS analysis was performed on day 2 on the non-CD25-depleted (B) and the CD25-depleted (C) MLR co-cultures to determine the percentage of CD4⁺FoxP3⁺ T-cells.

FIG. 3—MAPC mediate suppression via a soluble factor. A B6>BALB/c MLR plus MAPCs at 1:10 ratios were arranged by placing T-cells and stimulators in the lower well of a TransWell insert and MAPCs in the upper chamber, or by placing MAPCs in direct contact with stimulators and responders (A). (B) Supernatant taken from MAPC or control co-cultures on day 3 were added in 1:1 ratio with fresh media to B6>BALB/c MLR. Results of MAPCs at 1:10 and 1:100 ratios in direct contact with responding T-cells are shown for comparison. Proliferation was assessed using ³H-thymidine uptake as above. ELISA was performed on MLR supernatant harvested on the indicated day to determine the amount of proinflammatory (C) and anti-inflammatory (D) cytokines in culture with MAPCs at 1:10 and 1:100 ratios.

FIG. 4—MAPC inhibit T-cell allo-responses through the secretion of PGE2. B6>BALB/c MLR cultures were arranged as before. MAPCs, either untreated, treated overnight with 5 uM indomethacin to inhibit production of PGE2, or treated with vehicle were titrated in at 1:10 ratios. Proliferation was assessed as above (A).

FIG. 5—The capacity of MAPCs to delay GVHD mortality and limit target tissue destruction depends on anatomical location of the cells and their production of PGE2. BALB/c mice were lethally irradiated and then given 10⁶ BM cells from 136 mice on day 0 followed by 2×10⁶ purified CD25-depleted whole T-cells on day 2. On day 1, mice were given 5×10⁵ untreated B6 MAPCs or PBS delivered via intra-cardiac injections. Kaplan-Meier survival curve is representative of one experiment in which BM only and BM+T group had n=6, and MAPC group had n=8 (A). (B) BMT was performed as in (A) except mice were given PBS or 5×10⁵ MAPC intra-splenically (IS) on day 1. The survival curve is representative of 3 pooled experiments (BM only, n=18; BM-FT, n=20; MAPC, n=26) (MAPC vs. BM-FT, p<0.001). (C) Survival curve representative of one experiment in which mice received BMT plus untreated MAPC or MAPC pre-treated overnight with indomethacin before IS injection (BM only, n=5; BM+T, n=5; MAPC, n=10; MAPC indo, n=10) (MAPC vs. MAPC indo, p=0.002). Tissue taken from cohorts of mice from (B) were harvested on day 21 and embedded in OCT followed by freezing in liquid nitrogen, 6 uM sections were stained with H&E and analyzed for histopathological evidence of GVHD. Representative images are shown (D) (magnification×200). (E) The average GVHD score for BM only, BM+T, and BM+T+MAPC(IS) cohorts is shown. (F) Spleens were harvested from BMT plus MAPC IS transplanted mice on day 21 and snap frozen in OCT compound. Tissue sections were cut and stained using anti-luciferase and anti-POE synthase antibodies. Confocal analysis reveals that MAPC are found in the spleen at this time point and retain their ability to produce PGE2. 5F upper shows luciferase alone, 5F lower shows colocalization of PGE synthase with luciferase.

FIG. 6—MAPCs dampen T cell proliferation and activation within the local environment. “In vivo MLR” was performed by administering lethally irradiated BALB/c mice with 5×10⁵ B6 MAPCs IS (day 0) followed by 15×10⁶ B6 CFSE-labeled CD25-depleted T-cells (i.v.) (day 1). Control mice were given labeled T-cells alone plus sham surgeries. Spleens and LN were harvested on day 4 and analyzed via FACS for CD4 and CD8 expression and percent CFSE dilution (A). The proliferative capacity for CD4⁺ and CD8⁺ T-cells in the spleen (B) and LN(C) of transplanted mice was calculated as previously published¹⁸. (D, E) Activation markers for CD4⁺ and CD8⁺ T-cells in the spleen and LN were analyzed using FACS and graphed.

FIG. 7—MAPCs affect costimulatory molecule expression on T-cells and DCs in the spleen. FACS analysis of spleen cells harvested from transplanted mice on day 4 was performed to determine the percentage of CD4⁺ (A), CD8⁺ (B), and CD11c⁺ (C) cells that expressed the indicated co-stimulatory molecules. In this transplant, MAPCs were untreated or pre-treated with indomethacin, as described, before their application.

FIG. 8 (“Supplemental 1”)—Characterization of MAPC. (A) MAPCs isolated from 136 mice were differentiated into cells of mesodermal lineage and functionally tested for their ability to produce lipid droplets (adipocytes, Oil Red O), calcium deposition (osteocytes, Alizarin Red S), and accumulate collagen (chondrocytes, alkaline phosphatase). (B) Undifferentiated MAPCs (insert) or MAPCs directed to differentiate in vitro into cells representative of three germ layers, were stained for expression of CD31 and VWF (endothelium), HNF and albumin (endoderm), and GFAP and NF200 (neuroectoderm) and analyzed using confocal microscopy. In all images (except HNF) nuclear staining using DAPI is visualized as blue. (C) Undifferentiated MAPCs were examined for their expression of specific surface markers using flow cytometry. Isotype controls are shaded red. (D) RNA was isolated and cDNA was synthesized from undifferentiated MAPCs and murine embryonic stem cells. RT-PCR was used to examine the expression of Oct3/4 and Rex-1. No Template Control (NTC) and HPRT served as negative and positive controls, respectively. (E) Chromosomal analysis of MAPC shows a normal 40, XX karyotype.

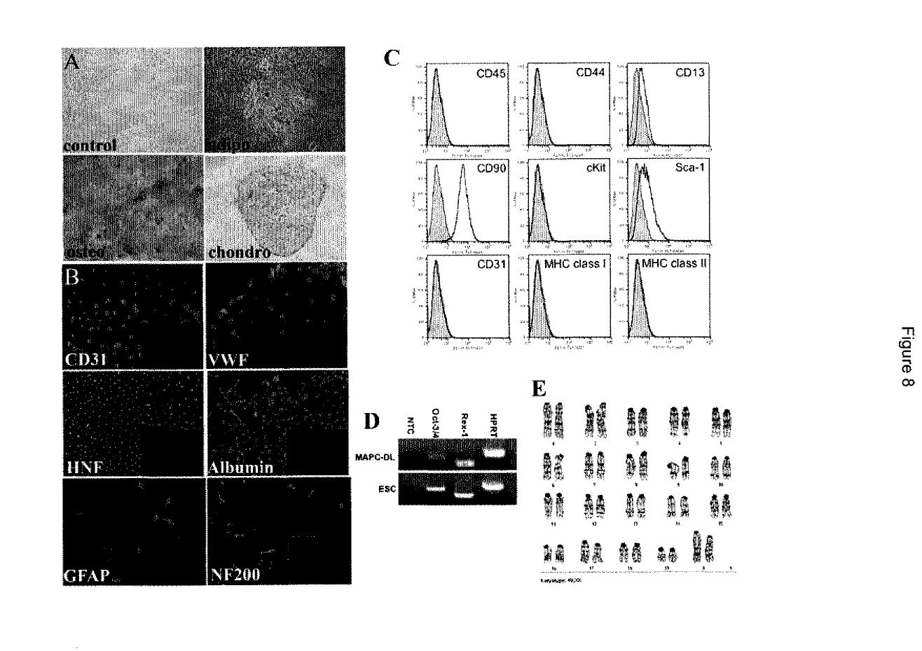

FIG. 9 (“Supplemental 2”)—MAPC inhibit ongoing allo-responses (JPG, 18 KB). B6>BALB/c MLR cultures were performed and B6 MAPCs were titrated in at 1:10 ratios on days 0, 1, 2, and 3. Cultures were pulsed on the indicated days and harvested 16 hours later. Proliferation was assessed as a measure of ³H-thymidine uptake.

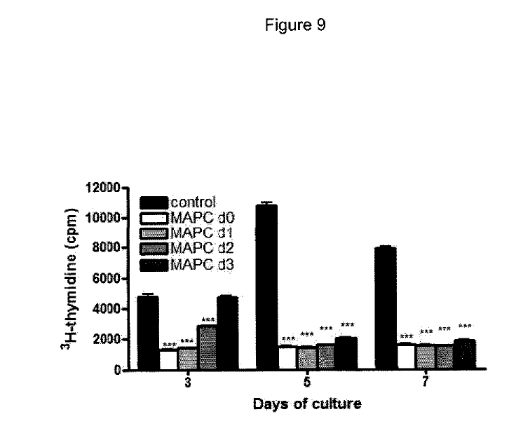

FIG. 10 (“Supplemental 3”)—Effects of PGE2 and IDO on T-cell proliferation (JPG, 68 KB). (A) RT-PCR verified that MAPC express PGE synthase. No template control (NTC) and HPRT were used as negative and positive controls. (B) Supernatant from MAPC cultures or MAPCs pretreated with 5 μM indomethacin overnight and washed were analyzed for the production of PGE2 via ELISA (Day 7 shown). (C) MLR cocultures were arranged using 200 μM 1-methyl tryptophan and 5 μM indomethacin in the culture media either alone or in combination to assess the contribution of IDO and PGE2 on MAPC mediated suppression, respectively. MLR were pulsed with ³H-thymidine on the indicated days and harvested 16 hours later.

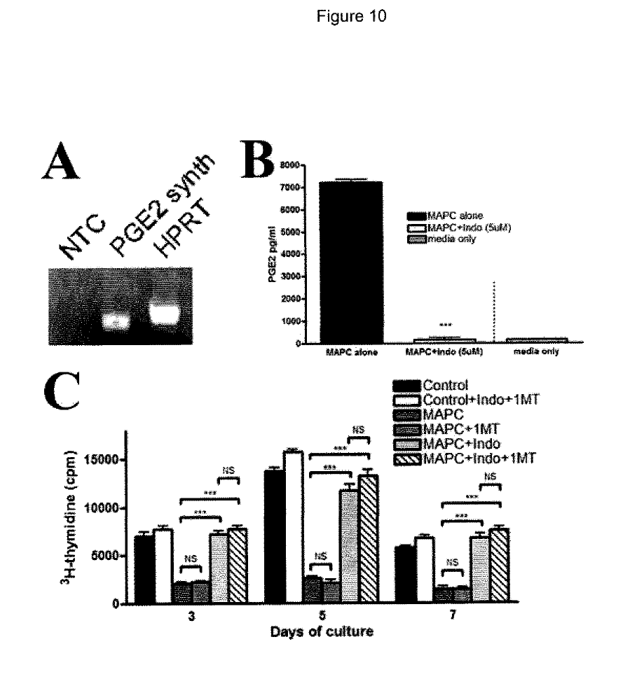

FIG. 11 (“Supplemental 4”)—Persistence of MAPC delivered intra-splenically (JPG, 168 KB). (A) BALB/c mice were lethally irradiated and given 10⁶ T-cell-depleted BM cells plus 5×10⁵ MAPC-DL IS. Individual mice and organs (top left-GI tract, top right-spleen, middle left-lung, middle right-LN, bottom left-femur, bottom right-liver) were monitored using bioluminescent imaging to determine the location of MAPCs on day 2, week 1, week 2, and week 3. (B) Weight curve from mice in FIG. 5C.

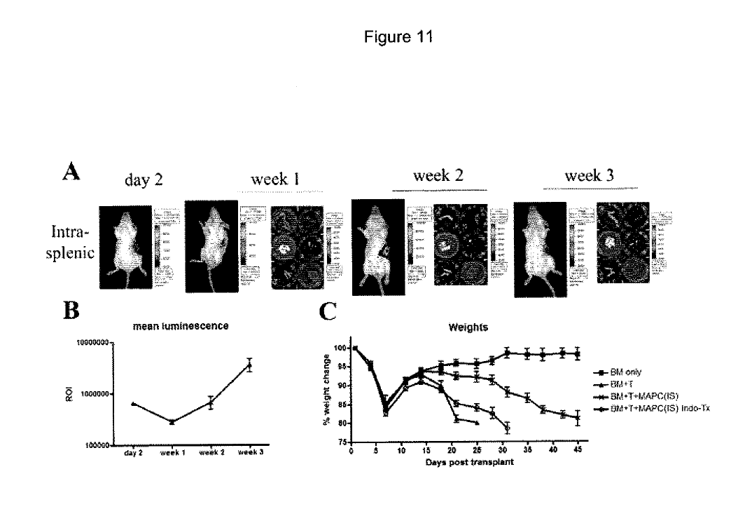

FIG. 12 (“Supplemental 5”)—Localization of MAPC to the spleen after “in vivo MLR” (PG, 78.3 KB). 5×10⁵ MAPC-DL were injected IS along with 15×10⁶ T cells. Day 4 bioluminescent imaging of spleens and lymph nodes from 6 mice revealed that MAPC remained within the spleen and had not migrated out to lymphoid tissue.

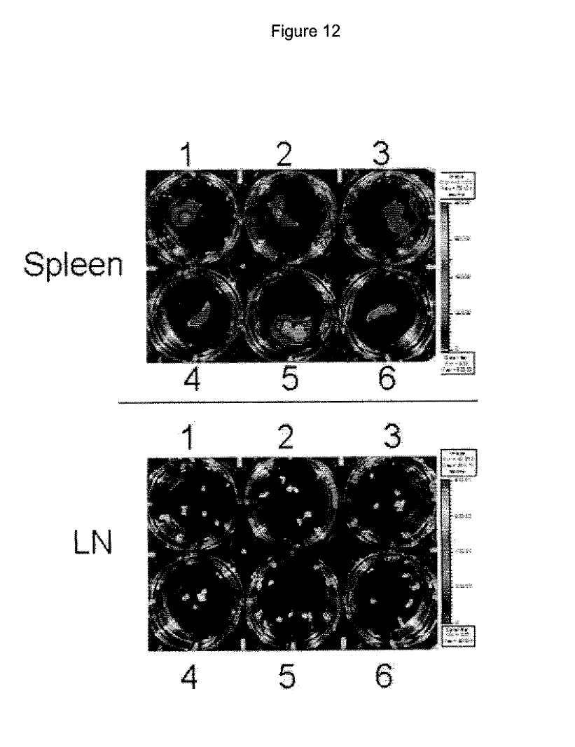

FIG. 13—MAPC synthesis of PGE2 in vitro is associated with the upregulation of negative co-stimulatory molecules and downregulation of positive costimulatory on T-cells and APCs.

### DETAILED DESCRIPTION OF THE INVENTION

It should be understood that this invention is not limited to the particular methodology, protocols, and reagents, etc., described herein and, as such, may vary. The terminology used herein is for the purpose of describing particular embodiments only, and is not intended to limit the scope of the disclosed invention, which is defined solely by the claims.

The section headings are used herein for organizational purposes only and are not to be construed as in any way limiting the subject matter described.

The methods and techniques of the present application are generally performed according to conventional methods well-known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane, Antibodies: A Laboratory Manual, Cold Spring Harbor Laboratory Press, Cold Spring Harbor, N.Y. (1990).

### DEFINITIONS

“A” or “an” means herein one or more than one; at least one. Where the plural form is used herein, it generally includes the singular.

A “cell bank” is industry nomenclature for cells that have been grown and stored for future use. Cells may be stored in aliquots. They can be used directly out of storage or may be expanded after storage. This is a convenience so that there are “off the shelf” cells available for administration. The cells may already be stored in a pharmaceutically-acceptable excipient so they may be directly administered or they may be mixed with an appropriate excipient when they are released from storage. Cells may be frozen or otherwise stored in a form to preserve viability. In one embodiment of the invention, cell banks are created in which the cells have been selected for enhanced expression of PGE2. Following release from storage, and prior to administration to the subject, it may be preferable to again assay the cells for potency, i.e., level of PGE2 expression. This can be done using any of the assays, direct or indirect, described in this application or otherwise known in the art. Then cells having the desired potency can then be administered to the subject for treatment.

“Co-administer” means to administer in conjunction with one another, together, coordinately, including simultaneous or sequential administration of two or more agents.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of and similar phrases using words of the root “comprise” are used herein as synonyms of “comprising” and have the same meaning.

“Comprised of” is a synonym of “comprising” (see above).

“Conditioned cell culture medium” is a term well-known in the art and refers to medium in which cells have been grown. Herein this means that the cells are grown for a sufficient time to secrete the factors that are effective to achieve any of the results described in this application, including reducing T-cell activation/proliferation, reducing pro-inflammatory cytokines, etc.

Conditioned cell culture medium refers to medium in which cells have been cultured so as to secrete factors into the medium. For the purposes of the present invention, cells can be grown through a sufficient number of cell divisions so as to produce effective amounts of such factors so that the medium has the effects, including reducing T-cell activation/proliferation, reducing pro-inflammatory cytokines, etc. Cells are removed from the medium by any of the known methods in the art, including, but not limited to, centrifugation, filtration, immunodepletion (e.g., via tagged antibodies and magnetic columns), and FACS sorting.

“EC cells” were discovered from analysis of a type of cancer called a teratocarcinoma. In 1964, researchers noted that a single cell in teratocarcinomas could be isolated and remain undifferentiated in culture. This type of stem cell became known as an embryonic carcinoma cell (EC cell).

“Effective amount” generally means an amount which provides the desired local or systemic effect, e.g., effective to ameliorate undesirable effects of inflammation, including reducing T-cell activation/proliferation, reducing pro-inflammatory cytokines, etc. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art. As used herein, “effective dose” means the same as “effective amount.”

“Effective route” generally means a route which provides for delivery of an agent to a desired compartment, system, or location. For example, an effective route is one through which an agent can be administered to provide at the desired site of action an amount of the agent sufficient to effectuate a beneficial or desired clinical result.

“Embryonic Stem Cells (ESC)” are well known in the art and have been prepared from many different mammalian species. Embryonic stem cells are stem cells derived from the inner cell mass of an early stage embryo known as a blastocyst. They are able to differentiate into all derivatives of the three primary germ layers: ectoderm, endoderm, and mesoderm. These include each of the more than 220 cell types in the adult body. The ES cells can become any tissue in the body, excluding placenta. Only the morula's cells are totipotent, able to become all tissues and a placenta. Some cells similar to ESCs may be produced by nuclear transfer of a somatic cell nucleus into an enucleated fertilized egg.

Use of the term “includes” is not intended to be limiting.

“Induced pluripotent stem cells (IPSC or IPS cells)” are somatic cells that have been reprogrammed. for example, by introducing exogenous genes that confer on the somatic cell a less differentiated phenotype. These cells can then be induced to differentiate into less differentiated progeny. IPS cells have been derived using modifications of an approach originally discovered in 2006 (Yamanaka, S. et al., Cell Stem Cell, 1:39-49 (2007)). For example, in one instance, to create IPS cells, scientists started with skin cells that were then modified by a standard laboratory technique using retroviruses to insert genes into the cellular DNA. In one instance, the inserted genes were Oct4, Sox2, Lif4, and c-myc, known to act together as natural regulators to keep cells in an embryonic stem cell-like state. These cells have been described in the literature. See, for example, Wernig et al., PNAS, 105:5856-5861 (2008); Jaenisch et al., Cell, 132:567-582 (2008); Hanna et al., Cell, 133:250-264 (2008); and Brambrink et al., Cell Stem Cell, 2:151-159 (2008). These references are incorporated by reference for teaching IPSCs and methods for producing them. It is also possible that such cells can be created by specific culture conditions (exposure to specific agents).

The term “isolated” refers to a cell or cells which are not associated with one or more cells or one or more cellular components that are associated with the cell or cells in vivo. An “enriched population” means a relative increase in numbers of a desired cell relative to one or more other cell types in vivo or in primary culture.

However, as used herein, the term “isolated” does not indicate the presence of only stem cells. Rather, the term “isolated” indicates that the cells are removed from their natural tissue environment and are present at a higher concentration as compared to the normal tissue environment. Accordingly, an “isolated” cell population may further include cell types in addition to stem cells and may include additional tissue components. This also can be expressed in terms of cell doublings, for example. A cell may have undergone 10, 20, 30, 40 or more doublings in vitro or ex vivo so that it is enriched compared to its original numbers in vivo or in its original tissue environment (e.g., bone marrow, peripheral blood, adipose tissue, etc.).

“MAPC” is an acronym for “multipotent adult progenitor cell”. It refers to a non-embryonic stem cell. It may give rise to cell lineages of more than one germ layer, such as two or all three germ layers (i.e., endoderm, mesoderm and ectoderm) upon differentiation. MAPCs may express one or more of telomerase, Oct 3/4 (i.e., Oct 3A), rex-1, rox-1 and sox-2, and SSEA-4. The term “adult” in MAPC is non-restrictive. It refers to a non-embryonic somatic cell. MAPCs are karyotypically normal and do not form teratomas in vivo. This acronym was first used in PCT/US2000/21387 to describe a pluripotent cell isolated from bone marrow. However, cells with pluripotential markers and/or differentiation potential have been discovered subsequently and, for purposes of this invention, may be equivalent to those cells first designated “MAPC.”

“Pharmaceutically-acceptable carrier” is any pharmaceutically-acceptable medium for the cells used in the present invention. Such a medium may retain isotonicity, cell metabolism, pH, and the like. It is compatible with administration to a subject in vivo, and can be used, therefore, for cell delivery and treatment.

The term “potency” refers to the ability of the cells (or conditioned medium from the cells) to achieve the various effects described in this application. Accordingly, potency refers to the effect at various levels, including, but not limited to, PGE2 levels that are effective for (1) reducing symptoms of inflammation; and/or (2) affecting underlying causes of inflammation such as reducing T-cell activation/proliferation, reducing pro-inflammatory cytokines, etc.

“Primordial embryonic germ cells” (PG or EG cells) can be cultured and stimulated to produce many less differentiated cell types.

“Progenitor cells” are cells produced during differentiation of a stem cell that have some, but not all, of the characteristics of their terminally-differentiated progeny. Defined progenitor cells, such as “cardiac progenitor cells,” are committed to a lineage, but not to a specific or terminally differentiated cell type. The term “progenitor” as used in the acronym “MAPC” does not limit these cells to a particular lineage. A progenitor cell can form a progeny cell that is more highly differentiated than the progenitor cell.

The term “reduce” as used herein means to prevent as well as decrease. In the context of treatment, to “reduce” is to either prevent or ameliorate one or more clinical symptoms. A clinical symptom is one (or more) that has or will have, if left untreated, a negative impact on the quality of life (health) of the subject. This also applies to the biological effects such as reducing T-cell activation/proliferation, reducing pro-inflammatory cytokines, etc., the end result of which would be to ameliorate the deleterious effects of inflammation.

“Selecting” a cell with a desired level of potency (e.g., for expressing and/or secreting PGE2) can mean identifying (as by assay), isolating, and expanding a cell. This could create a population that has a higher potency than the parent call population from which the cell was isolated.

To select a cell that expresses PGE2, would include both an assay to determine if there is PGE2 expression/secretion and would also include obtaining the expressor cell. The expressor cell may naturally express PGE2 in that the cell was not incubated with or exposed to an agent that induces PGE2 expression (for example, COX-1, COX-2, etc.). The cell may not be known to be a PGE2 expressor cell prior to conducting the assay.

Selection could be from cells in a tissue. For example, in this case, cells would be isolated from a desired tissue, expanded in culture, selected for PGE2 expression/secretion, and the selected cells further expanded.

Selection could also be from cells ex vivo, such as cells in culture. In this case, one or more of the cells in culture would be assayed for PGE2 expression/secretion and the cells obtained that express/secrete PGE2 could be further expanded.

Cells could also be selected for enhanced expression/secretion of PGE2. In this case, the cell population from which the enhanced expresser is obtained already expresses/secretes PGE2. Enhanced expression/secretion means a higher average amount (expression and/or secretion) of PGE2 per cell than in the parent PGE2 expressor population.

The parent population from which the higher expressor is selected may be substantially homogeneous (the same cell type). One way to obtain a higher expresser from this population is to create single cells or cell pools and assay those cells or cell pools for PGE2 expression/secretion to obtain clones that naturally express/secrete enhanced levels of PGE2 (as opposed to treating the cells with a PGE2 inducer) and then expanding those cells that are naturally higher expressors.

However, cells may be treated with one or more agents that will enhance PGE2 expression of the endogenous cellular PGE2 gene. Thus, substantially homogeneous populations may be treated to enhance expression.

If the population is not substantially homogeneous, then, it is preferable that the parental cell population to be treated contains at least 100 of the PGE2 expressor cell type in which enhanced expression is sought, more preferably at least 1,000 of the cells, and still more preferably, at least 10,000 of the cells. Following treatment, this sub-population can be recovered from the heterogeneous population by known cell selection techniques and further expanded if desired.

Thus, desired levels of PGE2 may be those that are higher than the levels in a given preceding population. For example, cells that are put into primary culture from a tissue and expanded and isolated by culture conditions that are not specifically designed to promote PGE2 expression, may provide a parent population. Such a parent population can be treated to enhance the average PGE2 expression per cell or screened for a cell or cells within the population that express higher PGE2 without deliberate treatment. Such cells can be expanded then to provide a population with a higher (desired) expression. In the exemplary material, stem cells are disclosed that secrete approximately 0.14 picogram/cell PGE2 on average. Enhanced expression for such cells could, therefore, be expression greater than about 0.14 picogram/cell PGE2 on average.

“Self-renewal” refers to the ability to produce replicate daughter stem cells having differentiation potential that is identical to those from which they arose. A similar term used in this context is “proliferation.”

“Stem cell” means a cell that can undergo self-renewal (i.e., progeny with the same differentiation potential) and also produce progeny cells that are more restricted in differentiation potential. Within the context of the invention, a stem cell would also encompass a more differentiated cell that has de-differentiated, for example, by nuclear transfer, by fusion with a more primitive stem cell, by introduction of specific transcription factors, or by culture under specific conditions. See, for example, Wilmut et al., Nature, 385:810-813 (1997); Ying et al., Nature, 416:545-548 (2002); Guan et al., Nature, 440:1199-1203 (2006); Takahashi et al., Cell, 126:663-676 (2006); Okita et al., Nature, 448:313-317 (2007); and Takahashi et al., Cell, 131:861-872 (2007).

Dedifferentiation may also be caused by the administration of certain compounds or exposure to a physical environment in vitro or in vivo that would cause the dedifferentiation. Stem cells also may be derived from abnormal tissue, such as a teratocarcinoma and some other sources such as embryoid bodies (although these can be considered embryonic stem cells in that they are derived from embryonic tissue, although not directly from the inner cell mass). Stem cells may also be produced by introducing genes associated with stem cell function into a non-stem cell, such as an induced pluripotent stem cell.

“Subject” means a vertebrate, such as a mammal, such as a human. Mammals include, but are not limited to, humans, dogs, cats, horses, cows, and pigs.

The term “therapeutically effective amount” refers to the amount of an agent determined to produce any therapeutic response in a mammal. For example, effective anti-inflammatory therapeutic agents may prolong the survivability of the patient, and/or inhibit overt clinical symptoms. Treatments that are therapeutically effective within the meaning of the term as used herein, include treatments that improve a subject's quality of life even if they do not improve the disease outcome per se. Such therapeutically effective amounts are readily ascertained by one of ordinary skill in the art. Thus, to “treat” means to deliver such an amount. Thus, treating can prevent or ameliorate any pathological symptoms of inflammation.

“Treat,” “treating,” or “treatment” are used broadly in relation to the invention and each such term encompasses, among others, preventing, ameliorating, inhibiting, or curing a deficiency, dysfunction, disease, or other deleterious process, including those that interfere with and/or result from a therapy.

### Stem Cells

The present invention can be practiced, preferably, using stem cells of vertebrate species, such as humans, non-human primates, domestic animals, livestock, and other non-human mammals. These include, but are not limited to, those cells described below.

Embryonic Stem Cells

The most well studied stem cell is the embryonic stem cell (ESC) as it has unlimited self-renewal and multipotent differentiation potential. These cells are derived from the inner cell mass of the blastocyst or can be derived from the primordial germ cells of a post-implantation embryo (embryonal germ cells or EG cells). ES and EG cells have been derived, first from mouse, and later, from many different animals, and more recently, also from non-human primates and humans. When introduced into mouse blastocysts or blastocysts of other animals, ESCs can contribute to all tissues of the animal. ES and EG cells can be identified by positive staining with antibodies against SSEA1 (mouse) and SSEA4 (human). See, for example, U.S. Pat. Nos. 5,453,357; 5,656,479; 5,670,372; 5,843,780; 5,874,301; 5,914,268; 6,110,739 6,190,910; 6,200,806; 6,432,711; 6,436,701, 6,500,668; 6,703,279; 6,875,607; 7,029,913; 7,112,437; 7,145,057; 7,153,684; and 7,294,508, each of which is incorporated by reference for teaching embryonic stem cells and methods of making and expanding them. Accordingly, ESCs and methods for isolating and expanding them are well-known in the art.

A number of transcription factors and exogenous cytokines have been identified that influence the potency status of embryonic stem cells in vivo. The first transcription factor to be described that is involved in stem cell pluripotency is Oct4. Oct4 belongs to the POU (Pit-Oct-Unc) family of transcription factors and is a DNA binding protein that is able to activate the transcription of genes, containing an octameric sequence called “the octamer motif” within the promoter or enhancer region. Oct4 is expressed at the moment of the cleavage stage of the fertilized zygote until the egg cylinder is formed. The function of Oct3/4 is to repress differentiation inducing genes (i.e., FoxaD3, hCG) and to activate genes promoting pluripotency (FGF4, Utf1, Rex1). Sox2, a member of the high mobility group (HMG) box transcription factors, cooperates with Oct4 to activate transcription of genes expressed in the inner cell mass. It is essential that Oct3/4 expression in embryonic stem cells is maintained between certain levels. Overexpression or downregulation of >50% of Oct4 expression level will alter embryonic stem cell fate, with the formation of primitive endoderm/mesoderm or trophectoderm, respectively. In vivo, Oct4 deficient embryos develop to the blastocyst stage, but the inner cell mass cells are not pluripotent. Instead they differentiate along the extraembryonic trophoblast lineage. Sall4, a mammalian Spalt transcription factor, is an upstream regulator of Oct4, and is therefore important to maintain appropriate levels of Oct4 during early phases of embryology. When Sall4 levels fall below a certain threshold, trophectodermal cells will expand ectopically into the inner cell mass. Another transcription factor required for pluripotency is Nanog, named after a celtic tribe “Tir Nan Og”: the land of the ever young. In vivo, Nanog is expressed from the stage of the compacted morula, is subsequently defined to the inner cell mass and is downregulated by the implantation stage. Downregulation of Nanog may be important to avoid an uncontrolled expansion of pluripotent cells and to allow multilineage differentiation during gastrulation. Nanog null embryos, isolated at day 5.5, consist of a disorganized blastocyst, mainly containing extraembryonic endoderm and no discernable epiblast.

Non-Embryonic Stem Cells

Stem cells have been identified in most tissues. Perhaps the best characterized is the hematopoietic stem cell (HSC). HSCs are mesoderm-derived cells that can be purified using cell surface markers and functional characteristics. They have been isolated from bone marrow, peripheral blood, cord blood, fetal liver, and yolk sac. They initiate hematopoiesis and generate multiple hematopoietic lineages. When transplanted into lethally-irradiated animals, they can repopulate the erythroid neutrophil-macrophage, megakaryocyte, and lymphoid hematopoietic cell pool. They can also be induced to undergo some self-renewal cell division. See, for example, U.S. Pat. Nos. 5,635,387; 5,460,964; 5,677,136; 5,750,397; 5,681,599; and 5,716,827. U.S. Pat. No. 5,192,553 reports methods for isolating human neonatal or fetal hematopoietic stem or progenitor cells. U.S. Pat. No. 5,716,827 reports human hematopoietic cells that are Thy-1⁺ progenitors, and appropriate growth media to regenerate them in vitro. U.S. Pat. No. 5,635,387 reports a method and device for culturing human hematopoietic cells and their precursors. U.S. Pat. No. 6,015,554 describes a method of reconstituting human lymphoid and dendritic cells. Accordingly, HSCs and methods for isolating and expanding them are well-known in the art.

Another stem cell that is well-known in the art is the neural stem cell (NSC). These cells can proliferate in vivo and continuously regenerate at least some neuronal cells. When cultured ex vivo, neural stem cells can be induced to proliferate as well as differentiate into different types of neurons and glial cells. When transplanted into the brain, neural stem cells can engraft and generate neural and glial cells. See, for example, Gage F. H., Science, 287:1433-1438 (2000), Svendsen S. N. et al, Brain Pathology, 9:499-513 (1999), and Okabe S. et al., Mech Development, 59:89-102 (1996). U.S. Pat. No. 5,851,832 reports multipotent neural stem cells obtained from brain tissue. U.S. Pat. No. 5,766,948 reports producing neuroblasts from newborn cerebral hemispheres. U.S. Pat. Nos. 5,564,183 and 5,849,553 report the use of mammalian neural crest stem cells. U.S. Pat. No. 6,040,180 reports in vitro generation of differentiated neurons from cultures of mammalian multipotential CNS stem cells. WO 98/50526 and WO 99/01159 report generation and isolation of neuroepithelial stem cells, oligodendrocyte-astrocyte precursors, and lineage-restricted neuronal precursors. U.S. Pat. No. 5,968,829 reports neural stem cells obtained from embryonic forebrain. Accordingly, neural stem cells and methods for making and expanding them are well-known in the art.

Another stem cell that has been studied extensively in the art is the mesenchymal stem cell (MSC). MSCs are derived from the embryonal mesoderm and can be isolated from many sources, including adult bone marrow, peripheral blood, fat, placenta, and umbilical blood, among others. MSCs can differentiate into many mesodermal tissues, including muscle, bone, cartilage, fat, and tendon. There is considerable literature on these cells. See, for example, U.S. Pat. Nos. 5,486,389; 5,827,735; 5,811,094; 5,736,396; 5,837,539; 5,837,670; and 5,827,740. See also Pittenger, M. et al, Science, 284:143-147 (1999).

Another example of an adult stem cell is adipose-derived adult stem cells (ADSCs) which have been isolated from fat, typically by liposuction followed by release of the ADSCs using collagenase. ADSCs are similar in many ways to MSCs derived from bone marrow, except that it is possible to isolate many more cells from fat. These cells have been reported to differentiate into bone, fat, muscle, cartilage, and neurons. A method of isolation has been described in U.S. 2005/0153442.

Other stem cells that are known in the art include gastrointestinal stem cells, epidermal stem cells, and hepatic stem cells, which have also been termed “oval cells” (Potten, C., et al., Trans R Soc Lond B Biol Sci, 353:821-830 (1998), Watt, F., Trans R Soc Lond B Biol Sci, 353:831 (1997); Alison et al., Hepatology, 29:678-683 (1998).

Other non-embryonic cells reported to be capable of differentiating into cell types of more than one embryonic germ layer include, but are not limited to, cells from umbilical cord blood (see U.S. Publication No. 2002/0164794), placenta (see U.S. Publication No. 2003/0181269, umbilical cord matrix (Mitchell, K. E. et al., Stem Cells, 21:50-60 (2003)), small embryonic-like stem cells (Kucia, M. et al., J Physiol Pharmacol, 57 Suppl 5:5-18 (2006)), amniotic fluid stem cells (Atala, A., J Tissue Regen Med, 1:83-96 (2007)), skin-derived precursors (Toma et al., Nat Cell Biol, 3:778-784 (2001)), and bone marrow (see U.S. Publication Nos. 2003/0059414 and 2006/0147246), each of which is incorporated by reference for teaching these cells.

Strategies of Reprogramming Somatic Cells

Several different strategies such as nuclear transplantation, cellular fusion, and culture induced reprogramming have been employed to induce the conversion of differentiated cells into an embryonic state. Nuclear transfer involves the injection of a somatic nucleus into an enucleated oocyte, which, upon transfer into a surrogate mother, can give rise to a clone (“reproductive cloning”), or, upon explantation in culture, can give rise to genetically matched embryonic stem (ES) cells (“somatic cell nuclear transfer,” SCNT). Cell fusion of somatic cells with ES cells results in the generation of hybrids that show all features of pluripotent ES cells. Explantation of somatic cells in culture selects for immortal cell lines that may be pluripotent or multipotent. At present, spermatogonial stem cells are the only source of pluripotent cells that can be derived from postnatal animals. Transduction of somatic cells with defined factors can initiate reprogramming to a pluripotent state. These experimental approaches have been extensively reviewed (Hochedlinger and Jaenisch, Nature, 441:1061-1067 (2006) and Yamanaka, S., Cell Stem Cell, 1:39-49 (2007)).

Nuclear Transfer

Nuclear transplantation (NT), also referred to as somatic cell nuclear transfer (SCNT), denotes the introduction of a nucleus from a donor somatic cell into an enucleated ogocyte to generate a cloned animal such as Dolly the sheep (Wilmut et al., Nature, 385:810-813 (1997). The generation of live animals by NT demonstrated that the epigenetic state of somatic cells, including that of terminally differentiated cells, while stable, is not irreversible fixed but can be reprogrammed to an embryonic state that is capable of directing development of a new organism. In addition to providing an exciting experimental approach for elucidating the basic epigenetic mechanisms involved in embryonic development and disease, nuclear cloning technology is of potential interest for patient-specific transplantation medicine.

Fusion of Somatic Cells and Embryonic Stem Cells

Epigenetic reprogramming of somatic nuclei to an undifferentiated state has been demonstrated in murine hybrids produced by fusion of embryonic cells with somatic cells. Hybrids between various somatic cells and embryonic carcinoma cells (Solter, D., Nat Rev Genet, 7:319-327 (2006), embryonic germ (EG), or ES cells (Zwaka and Thomson, Development, 132:227-233 (2005)) share many features with the parental embryonic cells, indicating that the pluripotent phenotype is dominant in such fusion products. As with mouse (Tada et al., Curr Biol, 11:1553-1558 (2001)), human ES cells have the potential to reprogram somatic nuclei after fusion (Cowan et al., Science, 309:1369-1373 (2005)); Yu et al., Science, 318:1917-1920 (2006)). Activation of silent pluripotency markers such as Oct4 or reactivation of the inactive somatic X chromosome provided molecular evidence for reprogramming of the somatic genome in the hybrid cells. It has been suggested that DNA replication is essential for the activation of pluripotency markers, which is first observed 2 days after fusion (Do and Scholer, Stem Cells, 22:941-949 (2004)), and that forced overexpression of Nanog in ES cells promotes pluripotency when fused with neural stem cells (Silva et al., Nature, 441:997-1001 (2006)).

Culture-Induced Reprogramming

Pluripotent cells have been derived from embryonic sources such as blastomeres and the inner cell mass (ICM) of the blastocyst (ES cells), the epiblast (EpiSC cells), primordial germ cells (EG cells), and postnatal spermatogonial stem cells (“maGSCsm” “ES-like” cells). The following pluripotent cells, along with their donor cell/tissue is as follows: parthogenetic ES cells are derived from murine oocytes (Narasimha et al., Curr Biol, 7:881-884 (1997)); embryonic stem cells have been derived from blastomeres (Wakayama et al., Stem Cells, 25:986-993 (2007)); inner cell mass cells (source not applicable) (Eggan et al., Nature, 428:44-49 (2004)); embryonic germ and embryonal carcinoma cells have been derived from primordial germ cells (Matsui et al., Cell, 70:841-847 (1992)); GMCS, maSSC, and MASC have been derived from spermatogonial stem cells (Guan et al., Nature, 440:1199-1203 (2006); Kanatsu-Shinohara et al., Cell, 119:1001-1012 (2004); and Seandel et al., Nature, 449:346-350 (2007)); EpiSC cells are derived from epiblasts (Brons et al., Nature, 448:191-195 (2007); Tesar et al., Nature, 448:196-199 (2007)); parthogenetic ES cells have been derived from human oocytes (Cibelli et al., Science, 295L819 (2002); Revazova et al., Cloning Stem Cells, 9:432-449 (2007)); human ES cells have been derived from human blastocysts (Thomson et al., Science, 282:1145-1147 (1998)); MAPC have been derived from bone marrow (Jiang et al., Nature, 418:41-49 (2002); Phinney and Prockop, Stem Cells, 25:2896-2902 (2007)); cord blood cells (derived from cord blood) (van de Ven et al., Exp Hematol, 35:1753-1765 (2007)); neurosphere derived cells derived from neural cell (Clarke et al., Science, 288:1660-1663 (2000)). Donor cells from the germ cell lineage such as PGCs or spermatogonial stem cells are known to be unipotent in vivo, but it has been shown that pluripotent ES-like cells (Kanatsu-Shinohara et al., Cell, 119:1001-1012 (2004) or maGSCs (Guan et al., Nature, 440:1199-1203 (2006), can be isolated after prolonged in vitro culture. While most of these pluripotent cell types were capable of in vitro differentiation and teratoma formation, only ES, EG, EC, and the spermatogonial stem cell-derived maGCSs or ES-like cells were pluripotent by more stringent criteria, as they were able to form postnatal chimeras and contribute to the germline. Recently, multipotent adult spermatogonial stem cells (MASCs) were derived from testicular spermatogonial stem cells of adult mice, and these cells had an expression profile different from that of ES cells (Seandel et al., Nature, 449:346-350 (2007)) but similar to EpiSC cells, which were derived from the epiblast of postimplantation mouse embryos (Brons et al., Nature, 448:191-195 (2007); Tesar et al., Nature, 448:196-199 (2007)).

Reprogramming by Defined Transcription Factors

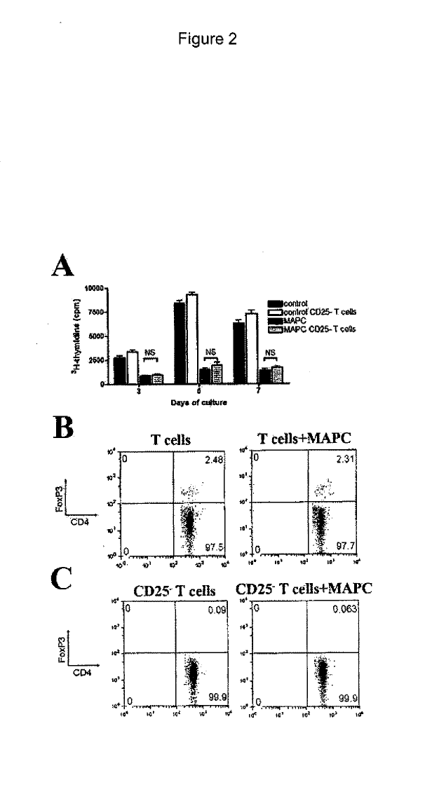

Takahashi and Yamanaka have reported reprogramming somatic cells back to an ES-like state (Takahashi and Yamanaka, Cell, 126:663-676 (2006)). They successfully reprogrammed mouse embryonic fibroblasts (MEFs) and adult fibroblasts to pluripotent ES-like cells after viral-mediated transduction of the four transcription factors Oct4, Sox2, c-myc, and Klf4 followed by selection for activation of the Oct4 target gene Fbx15 (FIG. 2A). Cells that had activated Fbx15 were coined iPS (induced pluripotent stem) cells and were shown to be pluripotent by their ability to form teratomas, although the were unable to generate live chimeras. This pluripotent state was dependent on the continuous viral expression of the transduced Oct4 and Sox2 genes, whereas the endogenous Oct4 and Nanog genes were either not expressed or were expressed at a lower level than in ES cells, and their respective promoters were found to be largely methylated. This is consistent with the conclusion that the Fbx15-iPS cells did not correspond to ES cells but may have represented an incomplete state of reprogramming. While genetic experiments had established that Oct4 and Sox2 are essential for pluripotency (Chambers and Smith, Oncogene, 23:7150-7160 (2004); Ivanona et al., Nature, 442:5330538 (2006); Masui et al., Nat Cell Biol, 9:625-635 (2007)), the role of the two oncogenes c-myc and Klf4 in reprogramming is less clear. Some of these oncogenes may, in fact, be dispensable for reprogramming, as both mouse and human iPS cells have been obtained in the absence of c-myc transduction, although with low efficiency (Nakagawa et al., Nat Biotechnol, 26:191-106 (2008); Waning et al., Nature, 448:318-324 (2008); Yu et al., Science, 318: 1917-1920 (2007)).

### MAPC

MAPC is an acronym for “multipotent adult progenitor cell” (non-ES, non-EG, non-germ). MAPC have the capacity to differentiate into cell types of at least two, such as, all three, primitive germ layers (ectoderm, mesoderm, and endoderm). Genes found in ES cells may also be found in MAPC (e.g., telomerase, Oct 3/4, rex-1, rox-1, sox-2). Oct 3/4 (Oct 3A in humans) appears to be specific for ES and germ cells. MAPC represents a more primitive progenitor cell population than MSC (Verfaillie, C. M., Trends Cell Biol 12:502-8 (2002), Jahagirdar, B. N., et al., Exp Hematol, 29:543-56 (2001); Reyes, M. and C. M. Verfaillie, Ann NY Acad Sci, 938:231-233 (2001); Jiang, Y. et al., Exp Hematol, 30896-904 (2002); and (Jiang, Y. et al., Nature, 418:41-9. (2002)).

Human MAPCs are described in U.S. Pat. No. 7,015,037 and U.S. application Ser. No. 10/467,963. MAPCs have been identified in other mammals, Murine MAPCs, for example, are also described in U.S. Pat. No. 7,015,037 and U.S. application Ser. No. 10/467,963. Rat MAPCs are also described in U.S. application Ser. No. 10/467,963.

These references are incorporated by reference for describing MAPCs first isolated by Catherine Verfaillie.

### Isolation and Growth of MAPCs

Methods of MAPC isolation are known in the art. See, for example, U.S. Pat. No. 7,015,037 and U.S. application Ser. No. 10/467,963, and these methods, along with the characterization (phenotype) of MAPCs, are incorporated herein by reference. MAPCs can be isolated from multiple sources, including, but not limited to, bone marrow, placenta, umbilical cord and cord blood, muscle, brain, liver, spinal cord, blood or skin. It is, therefore, possible to obtain bone marrow aspirates, brain or liver biopsies, and other organs, and isolate the cells using positive or negative selection techniques available to those of skill in the art, relying upon the genes that are expressed (or not expressed) in these cells (e.g., by functional or morphological assays such as those disclosed in the above-referenced applications, which have been incorporated herein by reference).

MAPCs from Human Bone Marrow as Described in U.S. Pat. No. 7,015,037

MAPCs do not express the common leukocyte antigen CD45 or erythroblast specific glycophorin-A (Gly-A). The mixed population of cells was subjected to a Ficoll Hypaque separation. The cells were then subjected to negative selection using anti-CD45 and anti-Gly-A antibodies, depleting the population of CD45⁺ and Gly-A⁺ cells, and the remaining approximately 0.1% of marrow mononuclear cells were then recovered. Cells could also be plated in fibronectin-coated wells and cultured as described below for 2-4 weeks to deplete the cells of CD45⁺ and Gly-A⁺ cells. In cultures of adherent bone marrow cells, many adherent stromal cells undergo replicative senescence around cell doubling 30 and a more homogenous population of cells continues to expand and maintains long telomeres.

Alternatively, positive selection could be used to isolate cells via a combination of cell-specific markers. Both positive and negative selection techniques are available to those of skill in the art, and numerous monoclonal and polyclonal antibodies suitable for negative selection purposes are also available in the art (see, for example, Leukocyte Typing V, Schlossman, et al., Eds. (1995) Oxford University Press) and are commercially available from a number of sources.

Techniques for mammalian cell separation from a mixture of cell populations have also been described by Schwartz, et al., in U.S. Pat. No. 5,759,793 (magnetic separation), Basch et al., 1983 (immunoaffinity chromatography), and Wysocki and Sato, 1978 (fluorescence-activated cell sorting).

Culturing MAPCs as Described in U.S. Pat. No. 7,015,037

MAPCs isolated as described herein can be cultured using methods disclosed herein and in U.S. Pat. No. 7,015,037, which is incorporated by reference for these methods.

Cells may be cultured in low-serum or serum-free culture medium. Serum-free medium used to culture MAPCs is described in U.S. Pat. No. 7,015,037. Many cells have been grown in serum-free or low-serum medium. In this case, the medium is supplemented with one or more growth factors. Commonly-used growth factors include but are not limited to bone morphogenic protein, basis fibroblast growth factor, platelet-derived growth factor, and epidermal growth factor. See, for example, U.S. Pat. Nos. 7,169,610; 7,109,032; 7,037,721; 6,617,161; 6,617,159; 6,372,210; 6,224,860; 6,037,174; 5,908,782; 5,766,951; 5,397,706; and 4,657,866; all incorporated by reference for teaching growing cells in serum-free medium.

### Additional Culture Methods

In additional experiments the density at which MAPCs are cultured can vary from about 100 cells/cm² or about 150 cells/cm² to about 10,000 cells/cm², including about 200 cells/cm² to about 1500 cells/cm² to about 2000 cells/cm². The density can vary between species. Additionally, optimal density can vary depending on culture conditions and source of cells. It is within the skill of the ordinary artisan to determine the optimal density for a given set of culture conditions and cells.

Also, effective atmospheric oxygen concentrations of less than about 10%, including about 1-5% and, especially, 3-5%, can be used at any time during the isolation, growth and differentiation of MAPCs in culture.

Cells may be cultured under various serum concentrations, e.g., about 2-20%. Fetal bovine serum may be used. Higher serum may be used in combination with lower oxygen tensions, for example, about 15-20%. Cells need not be selected prior to adherence to culture dishes. For example, after a Ficoll gradient, cells can be directly plated, e.g., 250,000-500,000/cm². Adherent colonies can be picked, possibly pooled, and expanded.

In one embodiment, used in the experimental procedures in the Examples, high serum (around 15-20%) and low oxygen (around 3-5%) conditions were used for the cell culture. Specifically, adherent cells from colonies were plated and passaged at densities of about 1.700-2300 cells/cm² in 18% serum and 3% oxygen (with PDGF and EGF).

In an embodiment specific for MAPCs, supplements are cellular factors or components that allow MAPCs to retain the ability to differentiate into all three lineages. This may be indicated by the expression of specific markers of the undifferentiated state. MAPCs, for example, constitutively express Oct 3/4 (Oct 3A) and maintain high levels of telomerase.

### Cell Culture

For all the components listed below, see U.S. Pat. No. 7,015,037, which is incorporated by reference for teaching these components.

In general, cells useful for the invention can be maintained and expanded in culture medium that is available and well-known in the art. Also contemplated is supplementation of cell culture medium with mammalian sera. Additional supplements can also be used advantageously to supply the cells with the necessary trace elements for optimal growth and expansion. Hormones can also be advantageously used in cell culture. Lipids and lipid carriers can also be used to supplement cell culture media, depending on the type of cell and the fate of the differentiated cell. Also contemplated is the use of feeder cell layers.

Cells in culture can be maintained either in suspension or attached to a solid support, such as extracellular matrix components. Stem cells often require additional factors that encourage their attachment to a solid support, such as type I and type II collagen, chondroitin sulfate, fibronectin, “superfibronectin” and fibronectin-like polymers, gelatin, poly-D and poly-L-lysine, thrombospondin and vitronectin. One embodiment of the present invention utilizes fibronectin. See, for example, Ohashi et al., Nature Medicine, 13:880-885 (2007); Matsumoto et al., J Bioscience and Bioengineering, 105:350-354 (2008); Kirouac et al., Cell Stem Cell, 3:369-381 (2008); Chua et al., Biomaterials, 26:2537-2547 (2005); Drobinskaya et al., Stem Cells, 26:2245-2256 (2008); Dvir-Ginzberg et al., FASEB J, 22:1440-1449 (2008); Turner et al., J Biomed Mater Res Part B: Appl Biomater, 82B:156-168 (2007); and Miyazawa et al., Journal of Gastroenterology and Hepatology, 22:1959-1964 (2007)).

Cells may also be grown in “3D” (aggregated) cultures. An example is PCT/US2009/31528, filed Jan. 21, 2009.

Once established in culture, cells can be used fresh or frozen and stored as frozen stocks, using, for example, DMEM with 40% FCS and 10% DMSO. Other methods for preparing frozen stocks for cultured cells are also available to those of skill in the art.

### Pharmaceutical Formulations

U.S. Pat. No. 7,015,037 is incorporated by reference for teaching pharmaceutical formulations. In certain embodiments, the cell populations are present within a composition adapted for and suitable for delivery, i.e., physiologically compatible.

In some embodiments the purity of the cells (or conditioned medium) for administration to a subject is about 100% (substantially homogeneous). In other embodiments it is 95% to 100%. In some embodiments it is 85% to 95%. Particularly, in the case of admixtures with other cells, the percentage can be about 10%-15%, 15%-20%, 20%-25%, 25%-30%, 30%-35%, 35%-40%, 40%-45%, 45%-50%, 60%-70%, 70%-80%, 80%-90%, or 90%-95%. Or isolation/purity can be expressed in terms of cell doublings where the cells have undergone, for example, 10-20, 20-30, 30-40, 40-50 or more cell doublings.

The choice of formulation for administering the cells for a given application will depend on a variety of factors. Prominent among these will be the species of subject, the nature of the condition being treated, its state and distribution in the subject, the nature of other therapies and agents that are being administered, the optimum route for administration, survivability via the route, the dosing regimen, and other factors that will be apparent to those skilled in the art. For instance, the choice of suitable carriers and other additives will depend on the exact route of administration and the nature of the particular dosage form.

Final formulations of the aqueous suspension of cells/medium will typically involve adjusting the ionic strength of the suspension to isotonicity (i.e., about 0.1 to 0.2) and to physiological pH (i.e., about pH 6.8 to 7.5). The final formulation will also typically contain a fluid lubricant.

In some embodiments, cells/medium are formulated in a unit dosage injectable form, such as a solution, suspension, or emulsion. Pharmaceutical formulations suitable for injection of cells/medium typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

The skilled artisan can readily determine the amount of cells and optional additives, vehicles, and/or carrier in compositions to be administered in methods of the invention. Typically, any additives (in addition to the cells) are present in an amount of 0.001 to 50 wt in solution, such as in phosphate buffered saline. The active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %.

In some embodiments cells are encapsulated for administration, particularly where encapsulation enhances the effectiveness of the therapy, or provides advantages in handling and/or shelf life. Cells may be encapsulated by membranes, as well as capsules, prior to implantation. It is contemplated that any of the many methods of cell encapsulation available may be employed.

A wide variety of materials may be used in various embodiments for microencapsulation of cells. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers.

Techniques for microencapsulation of cells that may be used for administration of cells are known to those of skill in the art and are described, for example, in Chang, P., et al., 1999; Matthew, et al., 1991; Yanagi, K., et al., 1989; Cai Z. H., et al., 1988; Chang, T. M., 1992 and in U.S. Pat. No. 5,639,275 (which, for example, describes a biocompatible capsule for long-term maintenance of cells that stably express biologically active molecules. Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of cells.

Certain embodiments incorporate cells into a polymer, such as a biopolymer or synthetic polymer. Examples of biopolymers include, but are not limited to, fibronectin, fibrin, fibrinogen, thrombin, collagen, and proteoglycans. Other factors, such as the cytokines discussed above, can also be incorporated into the polymer. In other embodiments of the invention, cells may be incorporated in the interstices of a three-dimensional gel. A large polymer or gel, typically, will be surgically implanted. A polymer or gel that can be formulated in small enough particles or fibers can be administered by other common, more convenient, non-surgical routes.

The dosage of the cells will vary within wide limits and will be fitted to the individual requirements in each particular case. In general, in the case of parenteral administration, it is customary to administer from about 0.01 to about 20 million cells/kg of recipient body weight. The number of cells will vary depending on the weight and condition of the recipient, the number or frequency of administrations, and other variables known to those of skill in the art. The cells can be administered by a route that is suitable for the tissue or organ. For example, they can be administered systemically, i.e., parenterally, by intravenous administration, or can be targeted to a particular tissue or organ; they can be administrated via subcutaneous administration or by administration into specific desired tissues.

The cells can be suspended in an appropriate excipient in a concentration from about 0.01 to about 5×10⁶ cells/ml. Suitable excipients for injection solutions are those that are biologically and physiologically compatible with the cells and with the recipient, such as buffered saline solution or other suitable excipients. The composition for administration can be formulated, produced, and stored according to standard methods complying with proper sterility and stability.

Administration into Lymphohematopoietic Tissues

Techniques for administration into these tissues are known in the art. For example, intra-bone marrow injections can involve injecting cells directly into the bone marrow cavity typically of the posterior iliac crest but may include other sites in the iliac crest, femur, tibia, humerus, or ulna; splenic injections could involve radiographic guided injections into the spleen or surgical exposure of the spleen via laparoscopic or laparotomy; Peyer's patches, GALT, or BALT injections could require laparotomy or laparoscopic injection procedures.

### Dosing

Doses for humans or other mammals can be determined without undue experimentation by the skilled artisan, from this disclosure, the documents cited herein, and the knowledge in the art. The dose of cells/medium appropriate to be used in accordance with various embodiments of the invention will depend on numerous factors. The parameters that will determine optimal doses to be administered for primary and adjunctive therapy generally will include some or all of the following: the disease being treated and its stage; the species of the subject, their health, gender, age, weight, and metabolic rate; the subject's immunocompetence; other therapies being administered; and expected potential complications from the subject's history or genotype. The parameters may also include: whether the cells are syngeneic, autologous, allogeneic, or xenogeneic; their potency (specific activity); the site and/or distribution that must be targeted for the cells/medium to be effective; and such characteristics of the site such as accessibility to cells/medium and/or engraftment of cells. Additional parameters include co-administration with other factors (such as growth factors and cytokines). The optimal dose in a given situation also will take into consideration the way in which the cells/medium are formulated, the way they are administered, and the degree to which the cells/medium will be localized at the target sites following administration.

The optimal dose of cells could be in the range of doses used for autologous, mononuclear bone marrow transplantation. For fairly pure preparations of cells, optimal doses in various embodiments will range from 10⁴ to 10⁸ cells/kg of recipient mass per administration. In some embodiments the optimal dose per administration will be between 10⁵ to 10⁷ cells/kg. In many embodiments the optimal dose per administration will be 5×10⁵ to 5×10⁶ cells/kg. By way of reference, higher doses in the foregoing are analogous to the doses of nucleated cells used in autologous mononuclear bone marrow transplantation. Some of the lower doses are analogous to the number of CD34⁺ cells/kg used in autologous mononuclear bone marrow transplantation.

In various embodiments, cells/medium may be administered in an initial dose, and thereafter maintained by further administration. Cells/medium may be administered by one method initially, and thereafter administered by the same method or one or more different methods. The levels can be maintained by the ongoing administration of the cells/medium. Various embodiments administer the cells/medium either initially or to maintain their level in the subject or both by intravenous injection. In a variety of embodiments, other forms of administration, are used, dependent upon the patient's condition and other factors, discussed elsewhere herein.

Cells/medium may be administered in many frequencies over a wide range of times. Generally lengths of treatment will be proportional to the length of the disease process, the effectiveness of the therapies being applied, and the condition and response of the subject being treated.

### Uses

Administering the cells is useful to reduce undesirable inflammation in any number of pathologies, including, but not limited to, colitis, alveolitis, bronchiolitis obliterans, ileitis, pancreatitis, glomerulonephritis, uveitis, arthritis, hepatitis, dermatitis, and enteritis.

Both IL-1 and COX-2 are known to upregulate PGE2. Accordingly, one or both of these can be admixed with the cells to be administered prior to administration or could be co-administered (simultaneous or sequential) with the cell. Administration, particularly of IL-1, may also include TNF.

In addition, other uses are provided by knowledge of the biological mechanisms described in this application. One of these includes drug discovery. This aspect involves screening one or more compounds for the ability to modulate the expression and/or secretion of PGE2 and/or the anti-inflammatory effects of the PGE2 secreted by the cells. This would involve an assay for the cell's ability express and/or secrete PGE2 and/or the anti-inflammatory effects of PGE2. Accordingly, the assay may be designed to be conducted in vivo or in vitro.

Cells (or medium) can be selected by directly assaying PGE2 protein or RNA. This can be done through any of the well-known techniques available in the art, such as by FACS and other antibody-based detection methods and PCR and other hybridization-based detection methods. Indirect assays may also be used for PGE2 expression, such as binding to any of the known PGE2 receptors (See, e.g., Kobayashi et al., Prostaglandins and Other Lipid Mediators, 68-69 (2002) 557-573; and Coleman et al., “Prostanoid Receptors EP1-EP4,” Pharmacological Reviews, 46:205-229). Indirect effects also include assays for any of the specific biological signaling steps/events triggered by PGE2 binding to any of its receptors. Therefore, a cell-based assay can also be used. These cells signaling steps have been described in Harris et al., above. PGE2 has also been shown to result in an increase in IL-4, IL-5, IL-10, 15LO, LXA4, and IL-6, and a decrease in TNFα, IFNγ, IL-2, IL-12, IL-12R, IL-1B, and IL-6. Accordingly, targets such these can also be used to assay for PGE2 expression/secretion.

Assays can also involve reducing activation and/or proliferation of CD4⁺ or CD8⁺ T-cells.

Assays for potency may be performed by detecting the factors modulated by PGE2. These may include IL-12, IL-2, IFN-γ, and TNF-α. Detection may be direct, e.g., via RNA or protein assays or indirect, e.g., biological assays for one or more biological effects of these factors.

Assays for expression/secretion of PGE2 include, but are not limited to, ELISA, Luminex. qRT-PCR, anti-PGE2 western blots, and PGE2 immunohistochemistry on tissue samples or cells.

Quantitative determination of PGE2 in cells and conditioned media can be performed using commercially available PGE2 assay kits (e.g., R&D Systems that relies on a two-step subtractive antibody-based assay).

A further use for the invention is the establishment of cell banks to provide cells for clinical administration. Generally, a fundamental part of this procedure is to provide cells that have a desired potency for administration in various therapeutic clinical settings.

Any of the same assays useful for drug discovery could also be applied to selecting cells for the bank as well as from the bank for administration.

Accordingly, in a banking procedure, the cells (or medium) would be assayed for the ability to achieve any of the above effects. Then, cells would be selected that have a desired potency for any of the above effects, and these cells would form the basis for creating a cell bank.

Cells can be isolated from a qualified marrow donor that has undergone specific testing requirements to determine that a cell product that is obtained from this donor would be safe to be used in a clinical setting. The mononuclear cells are isolated using either a manual or automated procedure. These mononuclear cells are placed in culture allowing the cells to adhere to the treated surface of a cell culture vessel. The MAPC cells are allowed to expand on the treated surface with media changes occurring on day 2 and day 4. On day 6, the cells are removed from the treated substrate by either mechanical or enzymatic means and replated onto another treated surface of a cell culture vessel. On days 8 and 10, the cells are removed from the treated surface as before and replated. On day 13, the cells are removed from the treated surface, washed and combined with a cryoprotectant material and frozen, ultimately, in liquid nitrogen. After the cells have been frozen for at least one week, an aliquot of the cells is removed and tested for potency, identity, sterility and other tests to determine the usefulness of the cell bank. These cells in this bank can then be used by thawing them, placing them in culture or use them out of the freeze to treat potential indications.

Another use is a diagnostic assay for efficiency and beneficial clinical effect following administration of the cells. Depending on the indication, there may be biomarkers available to assess.

A further use is to assess the efficacy of the cell to achieve any of the above results as a pre-treatment diagnostic that precedes administering the cells to a subject.

### Compositions

The invention is also directed to cell populations with specific potencies for achieving any of the effects described herein. As described above, these populations are established by selecting for cells that have desired potency. These populations are used to make other compositions, for example, a cell bank comprising populations with specific desired potencies and pharmaceutical compositions containing a cell population with a specific desired potency.

In one exemplified embodiment, cells are isolated and expanded without manipulating culture conditions or adding any agents for the purpose of increasing PGE2 expression. In this embodiment, cells secrete about 0.14 pg/cell PGE2 as assessed by an assay described in the Examples. Accordingly, in some embodiments, cells are selected for secretion of PGE2 above that number or manipulated in vitro to secrete PGE2 above that number. This includes, but is not limited to, amounts greater than about: 0.1, 0.2, 0.3, 0.4, 0.5, 0.6, 0.7, 0.8, 0.9, or 1.0 pg/cell or even greater.

### EXAMPLES

### Rationale/Background

The wider application of bone marrow transplant (BMT) has been limited, in part, by graft-versus-host disease (GVHD) complications. Human and mouse mesenchymal stem cells (MSCs) have been shown to suppress allogeneic-induced and nonspecific mitogen-induced T-cell proliferation in vitro (reviewed in detail^{1,2}). Implicated suppressive mechanisms have included IL-10³, TGF-β, hepatocyte growth factor⁴, indoleamine 2,3 dioxygenase (IDO)⁵, nitric oxide⁶, prostaglandin E2⁷, increased Tregulatory cells (Tregs)⁸, and activation of the PD-1 negative costimulatory pathway⁹. In vivo, there have been conflicting data regarding the potential of MSCs to suppress GVHD^{10,11,12}.

Non-hematopoietic stem cells, designated MAPC, can be co-purified with MSCs from bone marrow. MAPCs are generally believed to be a more primitive cell type than MSCs. MSCs kept for prolonged periods in culture tend to lose their differentiation capabilities and undergo senescence at ˜20-40 population doublings^{15,16}. In contrast to MSCs, MAPCs have an average telomere length remained constant for up to 100 population doublings in vitro¹³. Based upon their differential potential and reduced senescence, MAPCs have been considered as a potentially desirable non-hematopoietic stem cell source for use in allogeneic BMT. In fact, a multi-center phase I open label clinical trial of MultiStem®, based upon MAPC technology, was initiated in 2008.

For this Example, the inventor sought to determine whether MAPCs might be useful for GVHD prevention. They demonstrate that murine MAPCs are potently immune suppressive in vitro and can reduce GVHD lethality in vivo when present in the spleen, a site of initial allopriming, early post-BMT. Furthermore, they identify a mechanism of action for MAPCs to elicit T-cell inhibition and reduce GVHD-induced tissue injury in vivo.

### Results

MAPCs Inhibit T-Cell Proliferation and Activation.

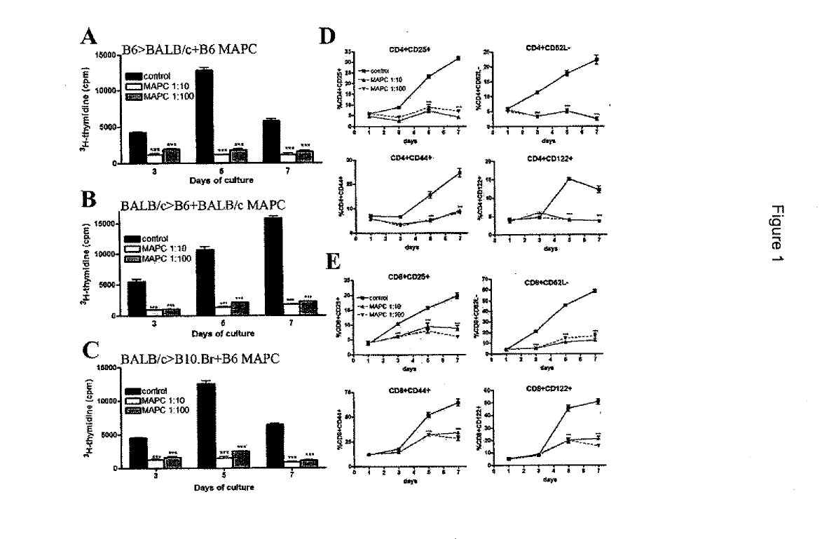

Murine MAPCs were expanded under low oxygen conditions and had tri-lineage differentiation potential²¹ (supplemental FIG. 1A, B). The murine MAPC preparations were CD45⁻, CD44⁻, CD13^{lo/+}, CD90⁺, c-kit⁻, Sca-1⁺, CD31⁻, MHC class I⁻, and MHC class II⁻ (supplemental FIG. 1C). RT-PCR expression analysis confirmed that these MAPCs expressed Oct-3/4 and Rex-1 (supplemental FIG. 1D). Tri-lineage differentiation potential, expression of Oct3/4 and Rex-1, and unique surface phenotype can distinguish MAPCs from other similar less primitive cell types such as MSCs. G-banding analysis revealed that 90% of the 20 metaphase cells analyzed had a normal karyotype (supplemental FIG. 1E). The remaining two cells had a tetraploid complement, one with an additional deletion within the long arm of chromosome 6. The tetraploid complement is within the normal limits for cultures that have been passaged several times. The finding of a single cell with a structural abnormality is considered a nonclonal event, and this cytogenetic study was interpreted as normal.

We have previously reported that MAPCs do not stimulate a T-cell alloresponse even when MAPCs have been pretreated with IFNγ to upregulate MHC class I, ICAM-1 and CD80 expression²². These studies did not address the possibility that MAPCs could actively suppress an immune response. To explore this possibility, B6 MAPCs were mixed with purified B6 T-cells and added to irradiated BALB/c stimulators. A significant reduction in proliferation was observed in MAPC-treated MLR at all time points (FIG. 1A). On the peak of the response (day 5), there was a near complete inhibition of T-cell alloresponses from MAPC co-cultures (91%, P<0.001 for 1:10; and 86%, P<0.001 for 1:100). To ensure the observed inhibitory effect was not dependent on this specific MAPC isolate or B6 strain of MAPC, BALB/c derived MAPCs were generated and mixed with BALB/c T-cells and B6 irradiated stimulators. The percent T-cell inhibition on the day of peak response was 88% (P<0.001) for 1:10 co-cultures, and 85% (P<0.001) for 1:100 co-cultures (FIG. 1B). These data indicated that the MAPC immune suppressive properties are not dependent on isolate or strain. The presence of MAPCs in MLR cultures significantly reduced the percentage of activated (CD25+, CD44hi, CD62Llo, CD122+) CD4⁺ (FIG. 1D) and CD8⁺ (FIG. 1E) T-effectors on days 5 and 7 (P<0.001 for both time points for 1:10 and 1:100 co-cultures). Taken together, the data show that MAPCs potently inhibit the activation and proliferation of alloresponsive T-cells in vitro.

To determine whether suppression was MHC-restricted, B6 MAPCs were added to purified BALB/c T-cells and irradiated B10.BR splenic stimulators (FIG. 1C). Third-party MAPCs potently inhibit allogeneic T-cell proliferation (89% and 80% average T-cell inhibition at peak for 1:10 and 1:100, respectively, P<0.001 for both), indicating inhibition was not MHC restricted. To determine if MAPCs had differential effects on resting vs. actively proliferating T-cells, MAPCs were added on days 0, 1, 2, and 3 to an MLR consisting of B6 T-cells and BALB/e stimulators. On both days 5 and 7, T-cell proliferation was significantly diminished by MAPC (P<0.001) (supplemental FIG. 2A), indicating that MAPCs can suppress an ongoing alloresponse.

Several studies attribute the T-cell inhibitory properties of MSCs to their ability to generate or regulate Tregs^{8,23,24}. MLR-MAPC co-cultures were performed using T-cells or CD25-depleted T-cells to determine if the lack of Tregs at the priming stage of the allogeneic response would impact MAPC-induced suppression. No difference was seen in the suppression potency between Treg-depleted vs. repleted cultures (FIG. 2A). Foxp3⁺Tregs percentages did not increase in MAPC- vs. control cultures through all time points (day 5 shown) in cultures using CD25-replete vs. depleted T-cells at all points (FIGS. 2B, C and data not shown). Thus, MAPC-mediated suppression does not depend upon Tregs in the responding T-cell fraction. Further, Tregs are not induced from the CD25⁻ T-cell fraction.

Murine MAPCs Mediate Suppression Via PGE2.

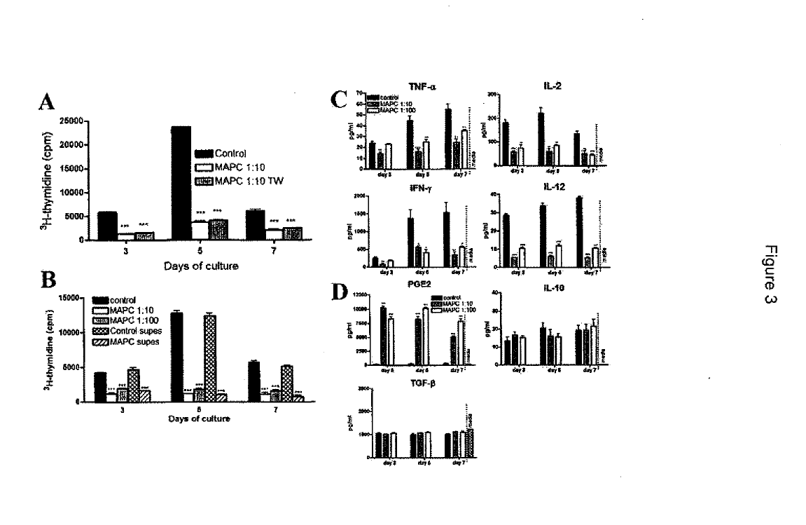

To determine if MAPCs suppress immune responses via release of soluble factors, the inventors utilized a TransWell co-culture system in which B6 T-cells and BALB/c splenic stimulators were placed in the lower chamber and B6-derived MAPCs were placed in the upper chamber of a TransWell. MAPCs inhibited T-cell alloresponses in a contact-independent manner (FIG. 3A), producing an 85% inhibition of T-cell proliferation on day 5 (P<0.01). No significant differences were seen due to the presence of a TransWell (P=0.19). To prove that MAPC-derived soluble factors were necessary and sufficient to induce immune suppression, cell-free supernatant from untreated and MAPC-treated MLR co-cultures were added in a 1:1 ratio with fresh media to a second MLR primary co-culture. MAPC-treated supernatant was equally as effective in inhibiting T-cell proliferation as MAPCs placed in direct contact with responders (FIG. 38; P=0.20). Supernatants were taken from B6>BALB/c MLR cultures and ELISAs were performed to determine effects on proinflammatory cytokine secretion. MAPCs decreased proinflammatory cytokine (TNFα, IL-12, IFN-γ, and IL-2) concentrations within these cultures at all time points (FIG. 3C). Although no significant increases in the amount of anti-inflammatory cytokines, IL-10 or TGF-β were observed, there was a significant increase in PGE2 concentrations within MAPC co-cultures (P<0.001) (FIG. 3D).

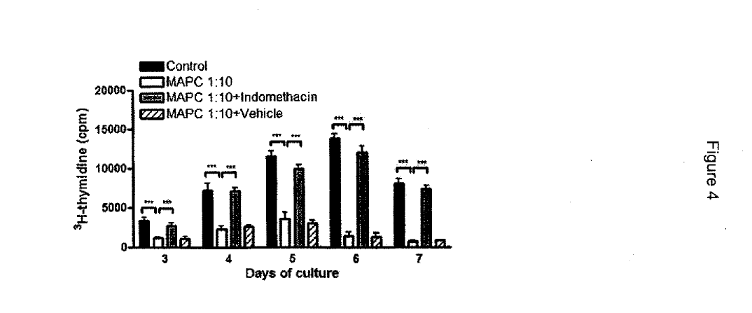

To determine if MAPCs were the source of PGE2 and if the increase in PGE2 was responsible for decreased T-cell alloresponsiveness, MAPCs expression of PGE2 synthase was verified, indicating they were capable of converting PGH2 into PGE2 (supplemental FIG. 3A). Moreover, analysis of supernatant taken from MAPC cultures alone had increased concentrations of PGE2 (8113±615 pg/ml at day 3, 7591±700 pg/ml at day 5) (data not shown). This indicated MAPCs are capable of producing PGE2 constitutively without the need for alto-stimulation. Overnight treatment of MAPCs with the COX1/2 inhibitor, indomethacin, potently inhibited the upstream synthesis of PGE2 for the period of the MLR culture (9 days) (supplemental FIG. 3B) without adversely affecting MAPC viability (data not shown), When indomethacin-treated MAPCs were added to MLR co-cultures, they no longer inhibited T-cell allo-responses (FIG. 4A). At the peak of the response (day 6), there was a 90% vs. 13% inhibition in allogeneic T-cell proliferation when untreated vs. indomethacin-pretreated MAPCs were in co-culture (P<0.001). At all other days examined, pre-treatment of MAPC with indomethacin lead to >90% restoration of proliferation.

MAPCs were found to upregulate IDO upon activation with IFN-γ (data not shown). To determine if IDO could account for the remaining inhibitory properties of these cells (˜10%), MAPCs were pre-treated with indomethacin and/or the MLR co-culture was treated with 1MT, a competitive inhibitor of IDO. The addition of 1MT to MLR co-cultures did not increase T-cell proliferation (supplemental FIG. 3C) and there was little/no additive effect of indomethacin pre-treatment of MAPCs with 1MT treatment of the co-culture (supplemental FIG. 3C).

Murine MAPC can Delay GVHD Mortality and Target Tissue Destruction if Localized to the Spleen Early Post-BMT.

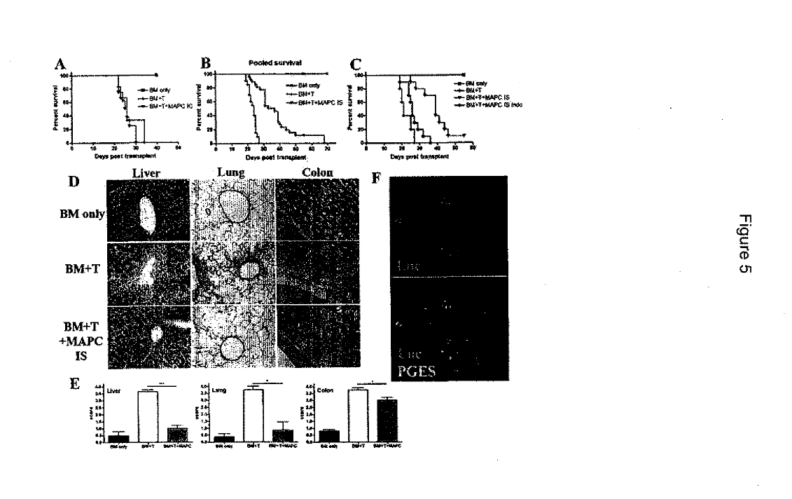

The prophylactic anti-GVHD efficacy of MAPC was tested in lethally irradiated BALB/c mice given B6 BM plus 2×10⁶ B6 CD25-depleted T-cells. Due to the lack of expression of MHC class 1 molecules on MAPCs, host mice were NK cell depleted on day −2 using anti-asialo GM-1 so as to ensure MAPCs were not rejected early post-transplant. Cohorts were given MAPC-DL or PBS via intra-cardiac (IC) injections directed toward the left-ventricle which allows for direct access to the systemic circulation and to a more widespread biodistribution and longer persistence of MAPC-DLs²². Despite their potent suppressive capacity in vitro, MAPC-treated mice vs. control mice had virtually identical survival rates (FIG. 5A). BLI of these mice revealed that most cells had migrated to BM cavities (skull, femur, spine), rather than to T-cell priming sites such as LNs or spleen (data not shown).

With the known short half life of PGE2 in vivo²², the inventors considered the possibility that sufficient quantities of this molecule might not be penetrating T-cell allopriming sites such as LNs and spleen. Subsequent studies were performed in which untreated or indomethacin-treated B6 MAPCs were given via an intra-splenic (IS) injection. Intra-splenic administration of MAPC was performed prior to the infusion of T-cells to allow time for MAPC-conditioning of the splenic microenvironment. Controls were given BM alone plus sham surgeries or BM plus T-cells and sham surgeries. BLI imaging of mice given MAPC-DL IS showed that these cells remained within the spleen for a period of up to 3 weeks (supplemental FIGS. 4A and 4B). It is known that SDF-1 (CXCL12) is upregulated in the spleen of mice following total body irradiation²⁵. Further, MAPCs express the receptor for this molecule (CXCR4). This interaction, therefore, may be responsible for the observed retention of MAPCs within the spleen. When compared to controls receiving BM+T-cells plus sham surgeries, mice given intra-splenic injections of untreated MAPCs have a significant improvement in survival (P<0.001), with two long-term survivors >55 days (FIG. 5B). MAPC-DLs present in the spleen continued to express PGE synthase as shown by co-staining (FIG. 5F). Therefore, although it is possible that some MAPCs may have undergone differentiation in vivo in this setting, they are still able to produce PGE2 as late as 3 weeks post-transplant. Indomethacin pretreatment of MAPCs precluded their protective effect (FIG. 5C) (MAPC vs. MAPC-Indo, P=0.0058), indicating that PGE2 is responsible for the suppressive potential of these cells in vivo. Mean body weights of these mice recapitulate these findings (supplemental FIG. 4C). On day 21 post-BMT, there was significantly more infiltrating lymphocytes in the liver and lung, resulting in increased necrotic foci and perivascular and peribronchiolar cuffing (FIG. 5D, E) along with large numbers of infiltrating lymphocytes in the colons of GVHD control vs. MAPC treated mice (FIG. 5D, E).

Therefore, MAPCs utilize PGE2 as a mechanism in vivo that leads to a significant increase in survival of mice with GVHD. In these experiments, the effects were dependent upon MAPC location.

MAPCs Diminish T-Cell Proliferation and Activation within the Local Environment.

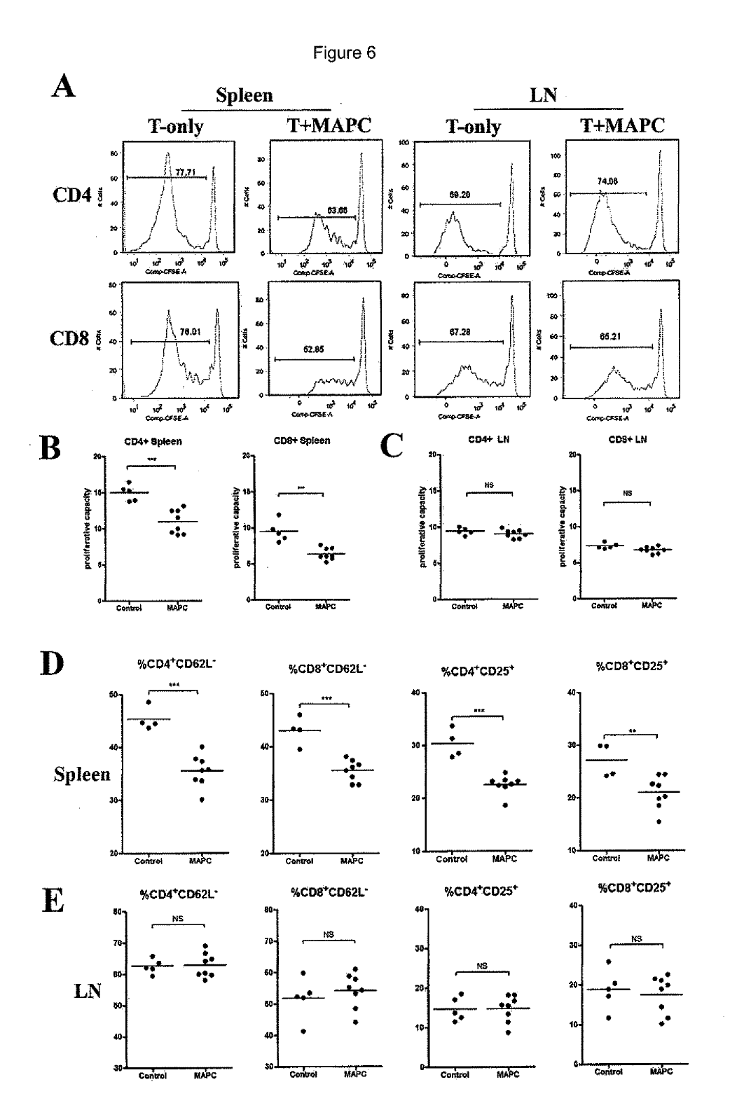

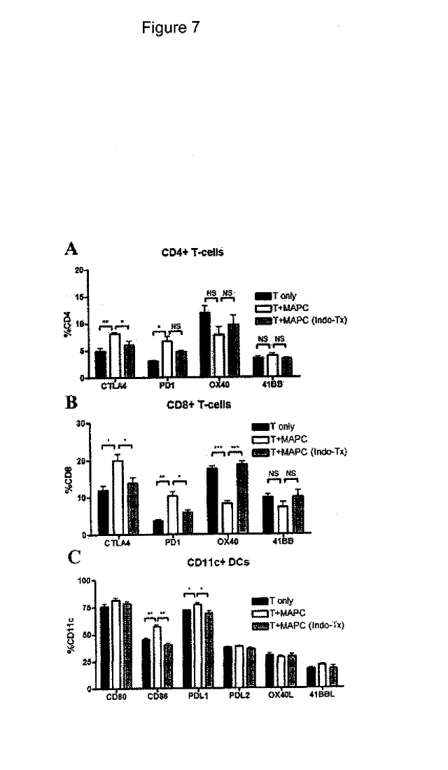

The direct in vivo effects of PGE2 on donor T-cell proliferation and activation using the MAPC intra-splenic administration model was determined. BALB/c mice were lethally irradiated and given B6 MAPC-DL via intra-splenic injections on day 0, and B6 CFSE-labeled CD25-depleted T-cells (15×10⁶) on day 1. Controls were given labeled T-cells alone plus sham injection. On day 4, LNs and spleens were analyzed by BLI. MAPCs were only located within the spleen and had not migrated out to the LNs (supplemental FIG. 5A). FACS analysis was performed to determine the percentage of T-cells that had divided during this time period and the proliferative capacity (the number of daughter cells that each responder cell produced) was calculated. There was a significantly reduced number of CD4⁺ and CD8⁺ T-cells that had undergone cellular division as determined by CFSE dilution in MAPC-treated vs. control groups (FIG. 6A). In the LN of the same mice, there were no significant differences in either CD4⁺ or CD8⁺ T-cells that had undergone cellular division. MAPCs resulted in a significantly reduced proliferative capacity of CFSE-labeled CD4⁺ and CD8⁺ T-cells in the spleen (FIG. 6B). Each alloreactive CD4 T-cell that had divided gave rise to 15 vs. 10 daughter cells in untreated vs. MAPC-treated mice (P=0.0005). Each alloreactive CD8 T-cell that divided gave rise to 10 vs. 6 daughter cells, respectively (P=0.0004) (FIG. 6B). In the LN of the same mice, there were no significant differences in CD4⁺ or CD8⁺ T-cell proliferative capacity between control- and MAPC-treated mice (FIG. 6C). Each CD4⁺ T-cell gave rise to an average of 9 and 9.4 daughter cells (P=0.25) and each CD8⁺ T-cell gave rise to 6.8 and 7.3 daughter cells in untreated vs. MAPC-treated groups (P=0.061). More splenic T-cells downregulated CD62L and upregulated CD25 in the control- vs. MAPC-treated group (FIG. 6D). In LNs, no such effects were observed (FIG. 6E), indicating that MAPCs limit allogeneic T-cell activation and expansion locally in vivo. Others have shown that PGE2 can influence the expression of co-stimulatory molecules²⁶. Therefore, the inventors tested T-cells and DCs within this in vivo MLR setting using treated or un-treated MAPCs to determine if the PGE2 effect on proliferation was due to its influence on co-stimulatory molecule expression. There were significantly more CD4⁺ and CD8⁺ T-cells within MAPC-treated groups than the control groups that expressed the negative co-stimulatory molecules PD-1 on CD4⁺ and CD8⁺ T-cells (FIG. 7A, B). Similarly, CTLA-4 was also expressed on a higher percentage of CD4⁺ and CD8⁺ T-cells in MAPC vs. control cultures. Also consistent with MAPC-induced suppression, the percentage of OX40⁺CD8⁺ T-cells was significantly lower than controls, along with a trend toward less OX40 and 41BB expression on CD4⁺ and CD8⁺ T-cells, respectively. These effects were reversed using MAPC treated with indomethacin prior to in vivo administration (FIG. 7). Within the MAPC vs. control groups, there was a significant increase in the percentage of DCs expressing PD-L1 and CD86 (FIG. 7C) without significant differences in the percentage of DCs expressing other costimulatory molecules (FIG. 7C). When using indomethacin-treated MAPCs, again, these effects were mostly reversed. There were no significant differences in the percentages of T-cells and APCs expressing ICOS, ICOS-L, CD40 and CD40L between groups (data not shown). Taken together, these data indicated that PGE2 accounts for the majority of the suppressive potential of MAPCs and has the downstream effect of increasing the percentage of cells expressing negative costimulatory regulators (PD1, PDL1, CTLA4), and decreasing the percentage of cells expressing positive costimulatory regulators (OX40, 41 BB).

### Materials and Methods

Mice.

BALB/c (H2^{d}), C57BL/6 (H2^{b})(termed B6) or B6-Ly5.2 (CD45 alleleic) mice were purchased from The Jackson Laboratory (Bar Harbor, Me.) or the National Institute of Health (Bethesda, Md.). B10.BR (H2^{k}) mice were purchased from The Jackson Laboratory. All mice were housed in specific pathogen-free facility in microisolator cages and used at 8-12 weeks of age in protocols approved by the Institutional Animal Care and Use Committee of the University of Minnesota.

MAPC Isolation and Culture.

MAPCs were isolated from B6 and BALB/c mice BM as described¹³. Briefly, BM was plated in DMEM/MCDB containing 10 ng/ml EGF (Sigma-Aldrich, St. Louis, Mo.), PDGF-BB (R&D systems, Minneapolis, Minn.), LIF (Chemicon International, Temecula, Calif.), 2% FCS (Hyclone, Waltham, Mass.), 1× selenium-insulin-transferrin-ethanolamine (SITE), 0.2 mg/ml linoleic acid-BSA, 0.8 mg/ml BSA, 1× chemically defined lipid concentrate, and 1× α-mercaptoethanol (all from Sigma-Aldrich). Cells were placed at 37 C in humidified 5% O₂, 5% CO₂ incubator. After 4 weeks, CD45⁺ and Ter119⁺ cells were depleted using MACS separation columns (Miltenyi Biotech, Auburn, Calif.) and plated at 10 cells/well for expansion. For in vivo experiments in which cells were tracked, MAPCs were used that stably express red fluorescent protein (DSred2) and firefly luciferase transgenes (termed MAPC-DL)¹⁷. For quality control, MAPCs were differentiated into cells representative of the mesodermal lineage, then subjected to in vitro trilineage (endothelium, endoderm, and neuroectodermal) differentiation to ensure multipotencyl³. MAPCs were analyzed for expression of CD90, Scat, CD45, CD44, CD13, cKit, CD31, MHC class I, and MHC class II, CD3, Mac1, B220, and Gr1 and tested for expression of transcription factors Oct3/4 and Rex-1 by RT-PCR. Twenty metaphase cells were evaluated by G-banding. Results are found in supplemental FIG. 1.

Mixed Leukocyte Reaction (MLR).

Lymph nodes (LNs) were harvested from B6 mice and T-cells were purified using by negative selection using PE-conjugated anti-CD19, anti-CD11c, anti-NK1.1 and anti-PE magnetic beads (Miltenyi Biotech). Purity was routinely >95%. Spleens were harvested from BALB/c mice, T-cell depleted (anti-Thy1.1), and irradiated (3000 cGy). B6 T-cells were mixed at a 1:1 ratio with BALB/c splenic stimulators and plated in a 96 well round bottom plate (10⁵ T-cells/well) or in the lower chamber of a 24 well plate TransWell insert (10⁶ T-cells/well). MAPCs were irradiated (3000 cGy) and plated in a 96 well round bottom plate (10⁴/well, 1:10) or in a 24 well TransWell plate (10⁵/well, 1:10). Cells were incubated in “T-cell media” in 200 μl/well (96 well) or 800 μl/well (24 well) of RPMI 1640 (Invitrogen, Carlsbad, Calif.) supplemented with 10% FCS, 50 mM 2-ME (Sigma-Aldrich), 10 mM HEPES buffer (Invitrogen), 1 mM sodium pyruvate (Invitrogen), amino acid supplements (1.5 mM L-glutamine, L-arginine, L-asparagine) (Sigma-Aldrich), 100 U/ml penicillin, 100 mg/ml streptomycin (Sigma-Aldrich). Cells were pulsed with ³H-thymidine (1 μCi/well) 16-18 hours prior to harvesting and counted in the absence of scintillation fluid on a β-plate reader. To inhibit PGE2 production, indomethacin (Sigma-Aldrich) resuspended in ethanol was diluted in T-cell media to reach a final concentration of 5 μM per flask and incubated overnight at 37 C, 5% CO2, The next day, cells were trypsinized and washed extensively with 2% FBS/PBS. Trypan blue exclusion was used to assess effects on live cells. In experiments evaluating the contribution of IDO, 1-methyl-D-tryptophan (1MT) (Sigma-Aldrich) was added to the culture media at a concentration of 200 μM.

Flow Cytometry.

Purified T-cells purified were stained with 1 μM carboxyfluorescein-succinimidyl-ester (CFSE, Invitrogen) for 2 minutes then washed. T-cells or CD11c⁺ DCs obtained from MLR cultures were stained for the expression of FoxP3, CD25, CD44, CD62L, CD122, PD-1, PDL1, PDL2, CTLA4, OX40, OX40L, 4-IBBL, 4-IBBL, ICOS, ICOSL, CD80, CD86, CD40L, or CD40 antigens. All antibodies were purchased through Pharmingen (San Diego, Calif.) or E-bioscience (San Diego, Calif.) and stained according to manufacturer's instructions then analyzed using FACSCalibur or FACSCanto (Becton Dickinson, San Jose, Calif.) and Flow Jo software (Treestar inc). Calculations to determine the proliferative capacity of T-cells were performed as described¹⁸.

Cytokines and PGE2 Quantification.

Quantitative determination of PGE2 in cell culture supernatants was performed using PGE2 assay kit (R&D Systems) by following manufacturer's instructions. Quantities of IL-10, TGF-β, IL-2, TNF-α, IFN-γ, and IL-12 were determined using Luminex technology (R&D Systems). The Parameter PGE2 Immunoassay is a 3.5 hour forward sequential competitive enzyme immunoassay designed to measure PGE2 in cell culture supernates, serum, plasma, and urine. This assay is based on the forward sequential competitive binding technique in which PGE2 present in a sample competes with horseradish peroxidase (HRP)-labeled PGE2 for a limited number of binding sites on a mouse monoclonal antibody. PGE2 in the sample is allowed to bind to the antibody in the first incubation. During the second incubation, HRP-labeled PGE2 binds to the remaining antibody sites. Following a wash to remove unbound materials, a substrate solution is added to the wells to determine the bound enzyme activity. The color development is stopped, and the absorbance is read at 450 nm. The intensity of the color is inversely proportional to the concentration of PGE2 in the sample.

In Vivo MLR¹⁹.

Host BALB/c stimulator mice were lethally irradiated using 850 cGy total body irradiation (TBI, ¹³⁷Cs), followed by intra-splenic injection of either PBS or 5×10⁵ MAPC. The next day, purified responder T-cells were labeled with 1 μM CFSE and 15×10⁶ cells were transferred into stimulator or syngeneic mice. After 96 hours, spleen and LNs were harvested for FACS analysis.

GVHD.

BALM recipients were lethally irradiated using 850 cGy TBI on day −1 followed by intra-splenic injection of either PBS or 5×10⁵ MAPC. On day 0, mice were infused intravenously (i.v.) with 10⁷ T-cell depleted (TCD) donor BM. On day +1, mice were given 2×10⁶ purified whole T-cells (CD4 and CD8) depleted of CD25. Recipient mice were NK-depleted with anti-asialo GM-1 (Wako Corp., Richmond, Va.) by intra-peritoneal (i.p.) injection of 25 on day −2, a dose previously determined to be highly effective for depletion of NK cells. Mice were monitored daily for survival and weighed twice weekly as well as examined for the clinical GVHD.

Tissue Histology.

On day 21, GVHD target organs (liver, lung, colon, skin, spleen) were harvested and snap-frozen in optimal cutting temperature (OCT) compound (Sakura, Tokyo, Japan) in liquid nitrogen. 6 μM sections were stained with hematoxylin and eosin and graded for GVHD using a semi-quantitative scoring system (0-4.0 grades in 0.5 increments)²⁰.

Immunofluorescence Microscopy.

Spleens taken from transplanted mice were embedded in OCT, snap-frozen in liquid nitrogen, and stored at −80° C. Cryosections (6 μM) were fixed in acetone for 10 min, air dried, and blocked with 1% BSA/PBS for 1 hour at room temperature. Primary antibody was diluted in 0.3% BSA/PBS and incubated for 2 hours. After 3 washes in PBS, sections were incubated with secondary antibody for 45 minutes. Sections were washed and mounted under a coverslip with 4,6-diamidino-2-phenylindole (DAPI) anti-fade solution (Invitrogen) and imaged on the following day at room temperature using an Olympus FluoView 500 Confocal Scanning Laser Microscope (Olympus, Center Valley, Pa.). Primary antibodies included anti-PGE synthase (Santa Cruz Biotechnology, Inc., Santa Cruz, Calif.) diluted 1:50, FITC-conjugated anti-Luciferase (Rockland Immunochemicals, Gilbertsville, Pa.) diluted 1:100. Goat-Cy3 secondary antibody (Jackson Immunoresearch Laboratories, West Grove, Pa.) was diluted to 1:200.

Bioluminescent Imaging (BLI) Studies.

A Xenogen IVIS imaging system (Caliper Life Sciences, Hopkinton, Mass.) was used for live animal imaging and imaging of organs taken from transplanted mice. MAPC-DL bearing mice were anesthetized with 0.25 mL Nembutol (1:10 diluted in PBS). Firefly luciferin substrate (0.1 mL, 30 mg/ml, Caliper Life Sciences, Hopkinton, Mass.) was injected i.p. or added to the media containing tissues and imaging was performed immediately after substrate addition. Data were analyzed and presented as photon counts per area.

Statistical Analysis.

The Kaplan-Meier product-limit method was used to calculate survival curve. Differences between groups in survival studies were determined using log-rank statistics. For all other data, a Student's t-test was used to analyze differences between groups, results were considered significant if the P value was <0.05.

### Conclusions

The study shows that MAPCs inhibit the proliferation and activation of allogeneic T-cells via the elaboration of PGE2. In viva, MAPCs did not home to lymphoid organs and did not suppress GVHD. Despite the contact-independence of MAPCs in suppressing alloresponse in vitro, MAPCs reduced GVHD only when injected into the spleen. The synthesis of PGE2 by MAPCs in situ resulted in an unfavorable in vivo environment for supporting T-cell activation. Thus, it is important to ensure homing of immunomodulatory cell types to relevant tissue sites to dampen T-cell priming and subsequent tissue injury.

The study shows that MAPCs are constitutive producers of PGE2. Inhibiting MAPC synthesis of PGE2 by treatment of the cells with a potent COX inhibitor restored in vitro T-cell proliferation in an allo-MLR culture to >90% of the control. In contrast, precluding other known inhibitors of an immune response, IL-10, TGF-β or IDO, using IL-10 receptor knock-out T-cells, anti-TGF-β antibodies or the competitive IDO inhibitor, 1MT, did not affect T-cell proliferation in MLR cultures (Supplemental FIG. 3C and data not shown). Thus, of the several known soluble factors that have been shown to contribute to the suppression of T-cells in MLR cultures in non-contact systems, PGE2 appears to be the dominant secreted molecule involved in MAPC-induced suppression of an in vitro alloresponse. Similarly, in vivo, inhibition of GVHD required MAPC production of PGE2 in situ.

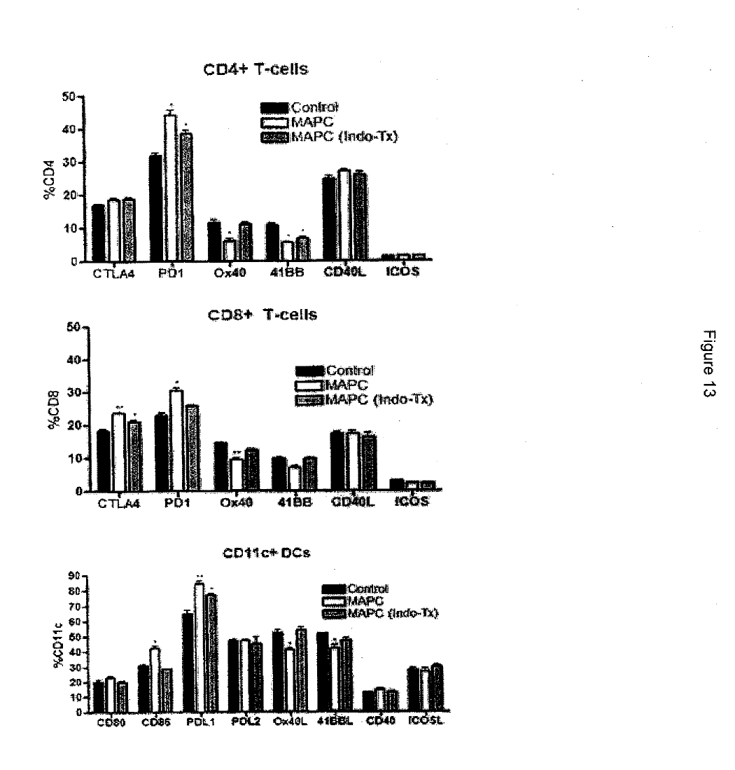

PGE2 can be produced by many cells²⁷ and influence the function of a wide array of immune cells including T-cells²⁸, B cells²⁹, macrophages³⁰, and DCs³¹. MAPC synthesis of PGE2 in vitro was associated with the upregulation of negative co-stimulatory molecules and downregulation of positive costimulatory on T-cells and APCs (FIG. 13). In contrast, a recent report has shown that human monocyte (CD14⁺) and myeloid (CD11c⁺) DCs upregulate positive costimulatory molecules (OX40L, CD70, 41BBL) if PGE2 is added during the maturation process³². We speculate that the apparent discordance may be due to the differences in the maturation status of the DCs at the time of PGE2 stimulation, although neither DC location (BM vs. spleen) nor species-specific differences can be excluded as explanations. PGE2 is known to have both stimulatory and inhibitory effects on DC activation, dependent upon the context in which PGE2 is encountered. DCs encountering PGE2 in the periphery have an increased activation and increased migratory abilities, whereas those encountering PGE2 within secondary lymphoid organs leads to decreased activation and decreased effector function³¹.

The inventors have shown³³ that donor MAPCs preferentially migrated to the BM after systemic delivery and are thus unlikely to directly interact with GVHD-causing donor T-cells within lymphoid organs. Because the half-life of PGE2 in vivo is extremely short (˜30 sec)³⁴, it is possible that this mechanism of MAPC-mediated suppression may not penetrate secondary lymphoid organs to a sufficient degree to inhibit T-cell activation and proliferation. MAPCs used in these studies did not express CD62L or CCR7, important for homing to secondary lymphoid organs (data not shown). To circumvent this problem, MAPCs were delivered directly into the spleens at the time of BMT, thereby restoring the capacity of MAPCs to suppress donor T-cell activation and proliferation in vivo. As predicted, this MAPC-mediated effect was only observed in the spleens and not in the LNs of transplanted mice (FIG. 6), confirming the initial hypothesis that PGE2 acts in a local manner. This suppressive effect on donor T-cells in vivo improved the survival of mice experiencing severe GVHD, a process that was almost entirely dependent on PGE2 production from MAPCs (FIG. 5B, C). Although the overall survival was improved by MAPC injection into the spleen, most mice eventually succumbed to the disease with only a minority becoming long-term survivors. Therefore, despite the fact that ˜8-fold more T-cells migrate to the spleen than to LNs during a given time point³³, T-cell activation within the LNs is sufficient, possibly along with residual T-cell activation within the spleen, to cause to lethal GVHD. The importance of secondary lymphoid organs for GVHD initiation can be derived from studies in which mice that lack all secondary lymphoid organs are incapable of developing severe GVHD^{36,37}. Because studies have shown that GVHD cannot be prevented by host splenectomy alone³⁸, our data suggest that MAPC-mediated suppression of donor T-cells within the spleen is not equivalent to a splenectomy. This may be due to the functional alterations of donor T-cells that are exposed to PGE2 within the spleen in lymphoid replete recipients in contrast to the unrestrained activation and proliferation of a higher number of donor T-cells that would traffic to the LNs of splenectomized hosts. Interestingly, MAPCs suppressed GVHD-induced tissue injury to a greater extent in the liver and lung as compared to the colon. Whether the influence of MAPCs on donor splenic T-cell function as evidenced by the pattern of costimulatory molecule expression or the homing of donor T-cells after exposure to MAPCs in the spleen would favor such a preferential organ-specific is unknown.

The requirement for homing of immune suppressive cells to secondary lymphoid organs to exert their maximum biological effect is not unique to MAPCs. For example, previous studies using murine BM-derived MSCs have proven to be ineffective in altering GVHD lethalityl². For Treg induced suppression of GVHD, high levels of CD62L expression was needed for optimal in vivo suppression of GVHD-induced lethality, though not for in vitro suppression^{39,40}. Whereas CCR5 expression on Tregs was not required for in vitro suppression, CCR5 knockout Tregs were inferior to wild-type Tregs in suppressing GVHD lethality in vivo, which was associated with a reduced accumulation of Tregs in lymphoid and non-lymphoid GVHD target organs beyond the first week post-BMT⁴¹. In solid organ allograft studies, Tregs suppression of graft rejection requires the migration of Tregs from the blood to the allograft to the draining LN⁴². Thus, the kinetics and homing patterns of immune modulatory cells to the sites of alloresponse are critical in determining the outcome of an alloresponse to foreign antigens and that GVHD inhibition by MAPCs requires homing to lymphoid sites that support GVHD initiation.

Although recent reports indicate that MAPCs can modify injury induced by vascular ischemia⁴³⁻⁴⁵, the in vitro and in vivo immunosuppressive properties of MAPCs remain to be further defined. One recent report has described the immunosuppressive potential of rat-derived MAPCs⁴⁶. Rat MAPCs inhibited alloresponses via a contact-independent mechanism. In contrast to the current study, MAPC-induced inhibition of T-cell alloproliferation in vitro was dependent upon IDO expression since 1MT reversed the suppressive effects of the rat MAPCs. Furthermore, the rat MAPCs expressed MHC class I antigens, in distinction to both human- and mouse-derived MAPCs that are targeted by NK-mediated lysis²². Although neither the homing receptor expression nor the in vivo homing or suppressor cell mechanisms responsible for GVHD inhibition were reported, the MAPCs reduced GVHD lethality.

The direct demonstration that PGE2 secretion is able to mediate donor T-cell suppression suggests a mechanism by which other cells may be able to suppress adverse alloresponses in vivo⁴⁷. Further, pharmacological strategies to achieve desired PGE2 concentrations in relevant target organs during the acute initiation phase may be useful for GVHD prevention. Approaches to target immune suppressive cells to allopriming sites may increase the efficacy of both non-hematopoietic stem cells and other immune suppressive populations to inhibit GVHD.

### REFERENCES

1. Fibbe W E, et al., Ann NY Acad Sci, 1106:272-278 (2007).
2. Uccelli A, Moretta L, Pistoia V., Eur J Immunol, 36:2566-2573 (2006).
3. Rasmusson I, et al., Exp Cell Res, 305:33-41 (2005).
4. Di Nicola M, et al., Blood, 99:3838-3843 (2002).
5. Meisel R, et al., Blood, 103:4619-4621 (2004).
6. Sato K, et al., Blood, 109:228-234 (2007).
7. Aggarwal S, Pittenger M F., Blood, 105:1815-1822 (2005).
8. Di Lanni M, et al., Exp Hematol, 36:309-318 (2008).
9. Augello A, et al., Eur J Immunol, 35:1482-1490 (2005).
10. Yanez R, et al., Stem Cells, 24:2582-2591 (2006).
11. Tisato V, et al., Leukemia, 21:1992-1999 (2007).
12. Sudres M, et al., J Immunol, 176:7761-7767 (2006).
13. Jiang Y, et al., Nature, 418:41-49 (2002).
14. Jiang Y, et al., Exp Hematol, 30:896-904 (2002).
15. Bruder S P et al., J Cell Biochem, 64:278-294 (1997).
16. Deans R J, Moseley A B., Exp Hematol, 28:875-884 (2000).
17. Tolar J et al., Mol Ther, 12:42-48 (2005).
18. Gudmundsdottir H, et al., J Immunol, 162:5212-5223 (1999).
19. Song H K, et al., Transplant Proc, 31:834-835 (1999).
20. Blazar B R, et al., Blood, 92:3949-3959 (1998).
21. Breyer A, et al., Exp Hematol, 34:1596-1601 (2006).
22. Tolar J, et al., Blood, 107:4182-4188 (2006).
23. Prevosto C, et al., Haematologica, 92:881-888 (2007).
24. Casiraghi F, et al., J Immunol, 181:3933-3946 (2008).
25. Ponomaryov T, et al., J Clin Invest, 106:1331-1339 (2000).
26. Takahashi H K, et al., J Immunol, 168:4446-4454 (2002).
27. Harris S G, et al., Trends Immunol, 23:144-150 (200).
28. Ruggeri P, et al., Immunopharmacol Immunotoxicol, 22:117-129 (2000).
29. Brown D M, et al., Clin Immunol Immunopathol, 63:221-229 (1992).
30. Ikegami R, et al., J Immunol, 166:4689-4696 (2001).
31. Harizi H, et al., Cell Immunol, 209:19-28 (2001).
32. Krause P, et al., Blood, 113:2451-2460 (2009).
33. Li H, et al., Stem Cells, 2008.
34. Fitzpatrick F A, et al., Prostaglandins, 19:917-931 (1980).
35. Pabst R, Westermann J., Scanning Microsc, 5:1075-1079; discussion 1079-1080 (1991).
36. Anderson B E, et al., Blood, 111:5242-5251 (2008).
37. Beilhack A, et al., Blood, 111:2919-2928 (2008).
38. Clouthier S G, et al., Transplantation, 73:1679-1681 (2002).
39. Taylor P A, et al., Blood, 104:3804-3812 (2004).
40. Ermann J, et al., Blood, 105:2220-2226 (2005).
41. Wysocki C A, et al., Blood, 106:3300-3307 (2005).
42. Zhang N, et al., “Regulatory T-cells Sequentially Migrate from Inflamed Tissues to Draining Lymph. Nodes to Suppress the Alloimmune Response” 30:458-469 (2009).
43. Aranguren X L, et al., J Clin Invest, 118:505-514 (2008).
44. Pelacho B, et al., J Tissue Eng Regen Med, 1:51-59 (2007).
45. Tolar J, et al., Exp Hematol, 35:682-690 (2007).
46. Kovacsovics-Bankowski M, et al., Cell Immunol (2008).
47. Ghansah T, et al., J Immunol, 173:7324-7330 (2004).

1. A method to treat inflammation in a subject, said method comprising selecting cells that have a desired potency for prostaglandin E2 expression and/or secretion; assaying said cells for a desired potency for prostaglandin E2 expression and/or secretion; and administering said cells having the desired potency for prostaglandin E2 expression and/or secretion to said subject in a therapeutically effective amount and for a time sufficient to achieve a therapeutic result, the cells being non-embryonic, non-germ cells that express one or more of oct4, telomerase, rex-1, or rox-1 and/or can differentiate into cell types of at least two of endodermal, ectodermal, and mesodermal germ layers.

2-15. (canceled)
